(19) United States
(12) Patent Application Publication
(10) Pub. No.: US 2025/0302369 A1
(43) Pub. Date: Oct. 2, 2025

(54) SYSTEMS AND METHODS FOR SEIZURE DETECTION

(71) Applicant: Ceribell, Inc., Sunnyvale, CA (US)

(72) Inventors: Archit GUPTA, Sunnyvale, CA (US); Suganya KARUNAKARAN, Sunnyvale, CA (US); Tanaya PURANIK, Cupertino, CA (US); Baharan KAMOUSI, Menlo Park, CA (US)

(21) Appl. No.: 19/098,805

(22) Filed: Apr. 2, 2025

Related U.S. Application Data
(60) Provisional application No. 63/573,290, filed on Apr. 2, 2024.

(51) Int. Cl. A61B 5/00; A61B 5/256; A61B 5/291; A61B 5/372; G16H 50/20

(52) U.S. Cl. CPC A61B 5/4094; A61B 5/256; A61B 5/291; A61B 5/372; A61B 5/6803; A61B 5/7264; A61B 5/7271; A61B 5/746; G16H 50/20; A61B 2560/0468

(57) ABSTRACT

Described herein are systems and methods for seizure detection. The systems may include a data module configured to obtain a plurality of electroencephalography (EEG) signals collected from a subject. The systems may also include a seizure detection module in communication with the data module configured to process and classify the data to detect various types of seizure activity using multiple classifiers. A control policy may be employed to determine a seizure burden on the aggregated seizure activity data and/or classifications. When the seizure burden is equal to or exceeds a threshold, a notification may be generated. The notification may be usable by a healthcare practitioner to assess whether the subject is having a seizure or at risk of having a seizure.

### CROSS-REFERENCE TO RELATED APPLICATIONS

This application claims priority to U.S. Provisional Application No. 63/573,290 filed on Apr. 2, 2024, which is hereby incorporated by reference in its entirely.

### FIELD

This application generally relates to systems and methods for seizure detection. The systems and methods may combine feature-based and deep learning techniques for differentiating between various types of seizure activity. EEG analysis using this hybrid technique may provide improved system performance by increasing the accuracy and speed of seizure detection.

### BACKGROUND

Seizures are neurological events characterized by abnormal electrical activity in the brain. Seizures may manifest with various intensities, may affect various regions of the brain (e.g., generalized seizures affect both sides of the brain, focal seizures affect one area of the brain), and may be associated with various conditions like epilepsy, brain tumors, stroke, and head injury. Thus, medical professionals must consider a myriad of factors when diagnosing and treating a patient experiencing seizures.

Seizures may also be characterized by a diverse range of neurological activity signals, which may be detected using sensors such as EEG (electroencephalography) sensors. The signals may be obtained from different regions of the brain and recorded as waveforms. EEGs are generally performed and analyzed by trained personnel, but may be difficult to evaluate given that seizures may manifest in various ways on an EEG recording. The EEG analysis process may also be time-consuming and subjective, leading to inter-observer variability. Moreover, reliance on visual analysis of EEG recordings may result in missing or misclassifying seizures, especially when detecting subtle or atypical seizure patterns. Another challenge with review of EEG recordings by medical personnel relates to the inability to detect seizures in real-time, which requires continuous monitoring and immediate recognition of abnormal EEG activity.

Various computing techniques including machine learning algorithms have been developed to analyze EEG signals and minimize expert intervention. However, the EEG signals provided to these machine learning algorithms require optimization to make the machine learning algorithms more effective in classifying the EEG data. Furthermore, these algorithms often lack the ability to accurately classify and/or detect various types of seizure activity (e.g., electrographic seizure and/or other abnormal EEG patterns such as highly pathological EEGs with a high likelihood of epileptiform activity).

Accordingly, it would be beneficial to have alternative systems and methods for classification of EEG signal data and seizure detection.

### SUMMARY

Described herein are systems and methods for detecting seizures. The systems and methods may employ multiple classifiers (e.g., multiple models) to differentiate between various types of seizure activity. For example, the systems and methods may utilize a hybrid model in which one or more feature-based models and one or more deep learning models are run sequentially or in parallel, and their output combined to produce an overall output (e.g., an overall classification). The hybrid model may detect seizures with greater sensitivity and specificity than when each model is run separately. Stated a different way, analysis of EEG data to detect seizures using the hybrid model may result in improved accuracy (e.g., a decrease of false positives) when compared to currently available seizure detection systems and methods. The improved accuracy in seizure detection and classification of seizure type may in turn result in earlier seizure detection as well as improved patient treatment given that seizure type may be more precisely diagnosed. The speed of seizure detection may also be improved (e.g., increased). In some variations, the hybrid model may detect status epilepticus with improved sensitivity and/or specificity.

The feature-based model may employ machine learning to improve the quality of the EEG signal by deriving features that focus on a distinct characteristic of an EEG signal, allowing the machine learning algorithm to more easily discern measured EEG signals and classify them. The deep learning model may include the use of a neural network, e.g., a convolutional neural network (CNN), to analyze and classify the EEG signals for seizure detection. The implementation of a control policy and seizure burden calculation after classification of the EEG signals may further help to provide improved accuracy of seizure detection. In some variations, the systems include a headband that may be worn by the subject comprising a plurality of electrodes from which the electroencephalography (EEG) signals are recorded.

The systems described herein may generally include one or more memories comprising program instructions and one or more processors (e.g., a central processing unit (CPU), a tensor processing unit (TPU), a graphics processing unit (GPU)). The one or more processors may cause, in response to retrieval and execution of the program instructions, operations including one or more of processing and pre-processing data obtained from a plurality of electroencephalography (EEG) signals recorded from a subject. For example, the one or more processors may pre-process electroencephalography (EEG) signals by filtering and dividing each of the plurality of EEG signals into a plurality of temporal segments. The one or more processors may then process the pre-processed EEG signals by extracting a plurality of features from each of the temporal segments, and determining a first output (e.g., a correction factor) based on a first probability value derived from the plurality of extracted features. Concurrently (or, in some instances, sequentially), the processed data may be transformed into a plurality of vectors and a second output (e.g., a second probability) determined based on the plurality of vectors, and a seizure classification determined for each of the temporal segments based on a combination of the first and second outputs. For example, the combination may include subtracting the first output (e.g., the correction factor) from the second output. In some variations, another correction factor may be generated based on detected artifacts (e.g., using feature values and electrode impedance) instead of from extracted features. One or both correction factors may be used when determining the seizure classification. For example, one or both correction factors may be subtracted from the second output. In some variations, if the second output is greater than a predetermined threshold after subtraction of a correction factor, the one or more processors of the system may, in response to the program instructions, determine the seizure classification for each temporal segment to be seizure-positive. The predetermined threshold may range from about 0.40 to about 0.70, including all values and sub-ranges therein. For example, the predetermined threshold range may be about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, or 0.70. If the second output is less than the predetermined threshold after subtraction of a correction factor therefrom, the one or more processors of the system may determine the seizure classification for each temporal segment to be seizure-negative. In some variations, the one or more processors of the system may also analyze data via a separate pathway (e.g., a different classifier/model) to determine seizure-like activity, e.g., highly pathological EEGs with a high likelihood of epileptiform activity.

In some variations, the systems for detecting seizures may include multiple modules. In these variations, the system may include a data module and a seizure detection module. The data module may be configured to receive data comprising a plurality of electroencephalography (EEG) signals recorded during a time window from a subject. The seizure detection module may include one or more processors (e.g., a central processing unit (CPU), a tensor processing unit (TPU), a graphics processing unit (GPU)) configured to pre-process the data by dividing each of the plurality of EEG signals into a plurality of temporal segments. The one or more processors (e.g., a central processing unit (CPU), a tensor processing unit (TPU), a graphics processing unit (GPU)) may also implement one or more machine learning models of a feature-based classifier configured to further process the data by extracting a plurality of features from each of the plurality of temporal segments, and generating a first output (e.g., a correction factor) based on a first probability value derived from the plurality of extracted features. The plurality of features that may be extracted may include at least one time-domain feature, at least one frequency-domain feature, and/or at least one time-frequency feature.

Additionally, the seizure detection module may include an encoder configured to transform the processed and/or pre-processed data into a plurality of vectors, and a decoder configured to determine a second probability value (e.g., a second output) based on the plurality of vectors. The one or more processors (e.g., a central processing unit (CPU), a tensor processing unit (TPU), a graphics processing unit (GPU)) may further be configured to determine a seizure classification for each of the temporal segments based on a combination of the first and second outputs (e.g., the correction factor and the second output). At least one of the encoder and the decoder may be part of a neural network, but in general both the encoder and the decoder are included. The neural network may include, but not be limited to, a convolutional neural network, a deep convolutional neural network, and a recurrent neural network. The encoder may include one or more attention blocks. The decoder may include a GRU (Gated Recurrent Unit), a LSTM (Long Short Term Memory) Unit, or a combination thereof.

In some variations, a spike detection module may be included as part of the system or as part of the seizure detection module. The spike detection module may be configured to identify one or more spikes within each of the plurality of temporal segments as a physiological spike or an artifactual spike based on one or more spike parameters, and classify each temporal segment as physiological or artifactual based on one or more spike metrics. The spike parameter may be a spike amplitude, a spike width, a spike prominence, or a spike polarity. The spike metric may be a spike frequency, a spike jitter, a spike polarity, and a spike count. In addition to spikes, the seizure detection module may further be configured to classify a temporal segment as an artifact when an impedance associated with an electrode used to record the EEG signals surpasses a threshold.

Some variations of the system may include a contextual classifier in addition to a feature-based classifier, an encoder, and a decoder. The contextual classifier may be configured to generate a plurality of context-based probability values based on a plurality of feature vectors extracted from a context window spanning a plurality of temporal segments. The plurality of temporal segments within the context window may comprise about 3 to about 30 temporal segments, including all values and sub-ranges therein. For example, the context window may include about 3 temporal segments, about 4 temporal segments, about 5 temporal segments, about 6 temporal segments, about 7 temporal segments, about 8 temporal segments, about 9 temporal segments, about 10 temporal segments, about 11 temporal segments, about 12 temporal segments, about 13 temporal segments, about 14 temporal segments, about 15 temporal segments, about 16 temporal segments, about 17 temporal segments, about 18 temporal segments, about 19 temporal segments, about 20 temporal segments, about 21 temporal segments, about 22 temporal segments, about 23 temporal segments, about 24 temporal segments, about 25 temporal segments, about 26 temporal segments, about 27 temporal segments, about 28 temporal segments, about 29 temporal segments, or about 30 temporal segments. In one variation, the contextual classifier may be a recurrent neural network.

Furthermore, the seizure detection modules described herein may include a decision module configured to combine two or more seizure probability values generated by a plurality of classifiers and correction factors to generate an aggregated probability value for each temporal segment. The aggregated probability value may correspond to a seizure burden and may be determined by one or more of a mean, a median, and a maximum of the seizure probability values generated by the plurality of classifiers (e.g., the feature-based classifier, the contextual classifier, and the decoder). The decision module may further be configured to compare a first moving average calculated over a first analysis window to a first threshold, and generate a seizure alert when the first moving average exceeds the first threshold. The first analysis window may range from about 5 minutes to about 60 minutes, including all values and sub-ranges therein. For example, the first analysis window may be about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some instances, the first moving analysis window may be about 5 minutes and the alert generated when the seizure burden equals or exceeds about 90%. Additionally or alternatively, the decision module may be configured to compare a second moving average calculated over a second time window to a second threshold, compare a third moving average calculated over a third analysis window to a third threshold, and generate a status epilepticus alert when both the second moving average equals or exceeds the second threshold and the third moving average equals or exceeds the third threshold. The second and third analysis windows may range from about 5 minutes to about 60 minutes, including all values and sub-ranges therein. For example, the second and third analysis windows may be about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some variations, the second analysis window may be about 10 minutes, the third moving analysis window may be about 60 minutes, the second threshold may be about 90%, and the third threshold may be about 20%.

The one or more processors (e.g., a central processing unit (CPU), a tensor processing unit (TPU), and a graphics processing unit (GPU)) may be further configured to subtract the correction factor from the second probability value (e.g., the second output). The seizure classification may be determined to be seizure-positive if, after subtracting the correction factor from the second probability value (e.g., the second output), the second probability value (e.g., the second output) is above a predetermined threshold. The predetermined threshold may range from about 0.40 to about 0.70, including all values and sub-ranges therein. For example, the predetermined threshold range may be about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, or 0.70. The seizure classification may be determined to be seizure-negative if, after subtracting the correction factor from the second probability value (e.g., the second output), the second probability value (e.g., the second output) is below the predetermined threshold. In some variations, a third module, e.g., a module for detecting seizure-like activity, may also be included in the system having a data module and a seizure detection module. The third module may be configured in a similar manner to the seizure detection module to determine whether EEG signals are seizure-like-positive or seizure-like-negative.

The data modules of the systems described herein may be configured to receive and record the plurality of EEG signals over one or more channels. For example, the one or more channels may include between 1 and 19 channels, including all values and sub-ranges therein. For example, the number of channels may be 1 channel, 2 channels, 3 channels, 4 channels, 5 channels, 6 channels, 7 channels, 8 channels, 9 channels, 10 channels, 11 channels, 12 channels, 13 channels, 14 channels, 15 channels, 16 channels, 17 channels, 18 channels, or 19 channels. In some variations, the one or more channels includes 8 channels. Each channel of the one or more channels may be assigned to an independent machine learning model, where for each channel, the extracted plurality of features may be applied to the machine learning model corresponding to each channel. The machine learning model may comprise one or more of a random forest, a boosted decision tree, a classification tree, a regression tree, a bagging tree, or a rotation forest.

Some variations of the systems for detecting seizures described herein may include one or more processors that may be configured to support one or more modules. The one or more modules may include a data module configured to receive data comprising a plurality of electroencephalography (EEG) signals recorded from a subject, and divide each of the plurality of EEG signals into a plurality of temporal segments; a spike detection module configured to analyze each of the plurality of temporal segments to identify one or more spikes, classify the one or more spikes as a physiological spike or an artifactual spike based on one or more spike parameters, and characterize each of the plurality of temporal segments as physiological or artifactual based on one or more spike metrics; a seizure detection module, where the seizure detection module may include a feature-based classifier, a transform model including an encoder and a decoder, and a contextual classifier; and a decision module configured to determine a seizure classification for each of the plurality of temporal segments based on an output of the seizure detection module.

The plurality of EEG signals may be recorded over a time window. A duration of the time window may range from about 1 second to about 10 minutes, including all values and sub-ranges therein. For example, the duration of the time window may be about 1 second, about 5 seconds, about 10 seconds, about 15 seconds, about 20 seconds, about 25 seconds, about 30 seconds, about 40 seconds, about 50 seconds, about 60 seconds, about 2 minutes, about 3 minutes, about 4 minutes, about 5 minutes, about 6 minutes, about 7 minutes, about 8 minutes, about 9 minutes, or about 10 minutes. The number of temporal segments included in the time window may vary, ranging from 2 to 30. For example, the time window may include 2, 3, 4, 5, 6, 7, 8, 9, 10, 11, 12, 13, 14, 15, 16, 17, 18, 19, 20, 21, 22, 23, 24, 25, 26, 27, 28, 29, or 30 temporal segments. In some variations, the time window includes three temporal segments. In other variations, the time window includes 30 temporal segments. The temporal segments may or may not be continuous temporal segments. Each of the temporal segments may range from about one second to about 20 seconds. In one variation, each temporal segment is about 10 seconds. In some variations, the duration of the time window may be about 5 minutes and include temporal segments of about 10 seconds.

Methods for detecting seizure in a subject are also described herein. The methods may generally include classifying temporal segments using a plurality of classification models to generate a plurality of seizure probability values, and combining the plurality of seizure probability values to determine the seizure classification for each temporal segment. More specifically, the methods may generally include obtaining data comprising a plurality of electroencephalography (EEG) signals recorded from electrodes disposed on a subject, extracting a plurality of features from each of the plurality of EEG signals to generate a first output using a feature-based model, transforming the data into a plurality of vectors using a transform model to generate a second output, and detecting seizure activity for each of the plurality of EEG signals by combining the first output and the second output. For example, combining may include subtracting the first output from the second output. The first output may be a correction factor. Extracting the plurality of features and transforming the data may be performed concurrently (e.g., in parallel) or sequentially. In one variation, extracting the plurality of features and transforming the data may be performed in parallel. As previously stated, the feature-based model may include one or more machine learning models, and the transform model may include a neural network and be comprised of at least one of an encoder and a decoder. After correction, if the second output is still above a predetermined threshold, the temporal segment may be classified as seizure-positive. As previously mentioned, the predetermined threshold may range from about 0.40 to about 0.70, including all values and sub-ranges therein.

The methods described herein may be used to analyze EEG signals for seizure activity, including at least one of seizures, seizure-like activity, electrographic seizure, status epilepticus, highly pathological EEGs with a high likelihood of epileptiform activity, post-ictal activity, and abnormal EEG patterns. In some instances, the methods may include diagnosing epilepsy in the subject based on the detected seizure activity and/or treating the subject for the detected seizure activity.

Other methods for detecting seizures may include receiving data comprising a plurality of electroencephalography (EEG) signals recorded from a subject during a time window, pre-processing the data by dividing each of the plurality of EEG signals into a plurality of temporal segments, extracting a plurality of features from each of the temporal segments, and generating a first output (e.g., a correction factor) based on a first probability value derived from the extracted plurality of features. The plurality of features that are extracted may include one or more of a time-domain feature, a frequency-domain feature, and a time-frequency feature. Determining the first probability value may include applying a random forest model to the plurality of extracted features to classify each temporal segment as seizure-positive or seizure-negative on a per-channel basis. The methods may further include transforming the pre-processed data into a plurality of vectors, determining a second probability value based on the plurality of vectors, and determining a seizure classification for each of the temporal segments based on a combination of the first and second probability values.

In some variations, the method may further include subtracting the correction factor from the second probability value when the first probability value is below a predetermined threshold. The predetermined threshold may range from about 0.40 to about 0.70, including all values and sub-ranges therein. The seizure classification may be determined to be seizure-positive if, after subtracting the correction factor, the second probability value is above the predetermined threshold. The seizure classification may be determined to be seizure-negative if, after subtracting the correction factor, the second probability value is below the predetermined threshold. Transforming the pre-processed data into the plurality of vectors may include encoding each temporal segment as a separate vector using a neural network. The neural network may be a convolutional neural network, a deep convolutional neural network, a recurrent neural network, or a combination thereof.

The methods described herein may also include using a spike detection module configured to analyze the plurality of temporal segments. In some variations, the spike detection module may be configured to identify one or more spikes within each of the plurality of temporal segments, classify the one or more spikes based on one or more spike parameters, and characterize the each temporal segment as physiological or artifactual based on one or more spike metrics. The spike parameter may be a spike amplitude, a spike width, a spike prominence, or a spike polarity. The spike metric may be a spike frequency, a spike jitter, a spike polarity, or a spike count. In addition to identifying, classifying, and characterizing spikes, the seizure detection module may further be configured to classify a temporal segment as an artifact when an impedance associated with an electrode used to record the EEG signals surpasses a threshold.

The methods may include analyzing relationships between adjacent temporal segments of the plurality of temporal segments to generate context-based probability values using a contextual classifier and/or processing the plurality of temporal segments in parallel using one or more attention blocks. In some instances, once the temporal segments have been classified, the methods may include combining seizure classifications over a moving analysis window and calculating a seizure burden based on the combined classifications. An alert may be generated when the seizure burden exceeds a predetermined threshold. In some variations, the seizure burden may be calculated by calculating a first moving average over a first moving analysis window, and comparing the first moving average to a first threshold. In this variation, the seizure burden alert may be generated when the first moving average exceeds the first threshold. The first analysis window may range from about 5 minutes to about 60 minutes, including all values and sub-ranges therein. For example, the first analysis window may be about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some variations, the first moving analysis window may be about 5 minutes and the alert may be generated when the seizure burden equals or exceeds about 90%. Additionally or alternatively, when a status epilepticus alert is to be generated, the seizure burden may be calculated by calculating a second moving average over a second moving analysis window, calculating a third moving average over a third moving analysis window, and comparing the second moving average to a second threshold and the third moving average to a third threshold. The status epilepticus alert may be generated when both the second moving average equals or exceeds the second threshold and the third moving average equals or exceeds the third threshold. The second and third analysis windows may range from about 5 minutes to about 60 minutes, including all values and sub-ranges therein. For example, the second and third analysis windows may be about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some variations, the second analysis window may be about 10 minutes, the third moving analysis window may be about 60 minutes, the second threshold may be about 90%, and the third threshold may be about 20%.

Other methods for detecting seizures may include receiving data comprising a plurality of electroencephalography (EEG) signals recorded from at least one electrode disposed on a subject/patient (e.g., an electrode of a headband worn on the head of a subject/patient), pre-processing the data by dividing each of the plurality of EEG signals into a plurality of temporal segments, and further processing the data by analyzing each of the plurality of temporal segments to identify one or more spikes. The one or more spikes may be classified as a physiological spike or an artifactual spike based on one or more spike parameters, and each of the plurality of temporal segments characterized as physiological or artifactual based on one or more spike metrics. In some instances, the one or more spikes may be used to further characterize the severity of artifacts for a temporal segment. A plurality of features may be extracted from each of the plurality of temporal segments to generate a first output using a feature-based classifier, and one or more deep learning models used in parallel (e.g., concurrently) or sequentially, to transform the data received from each of the plurality of temporal segments into a plurality of vectors using a transform model to generate a second output, and detecting seizure activity for each of the plurality of temporal segments based on at least the second output and a correction factor.

As previously mentioned, the plurality of electroencephalography (EEG) signals may recorded over one or more channels. The one or more channels may comprise between 8 and 19 channels, including all values and sub-ranges therein. For example, the number of channels may be 1 channel, 2 channels, 3 channels, 4 channels, 5 channels, 6 channels, 7 channels, 8 channels, 9 channels, 10 channels, 11 channels, 12 channels, 13 channels, 14 channels, 15 channels, 16 channels, 17 channels, 18 channels, or 19 channels. In some variations, the one or more channels may be 8 channels. Each channel of the one or more channels may be assigned to an independent machine learning model, wherein for each channel, the extracted plurality of features is applied to the machine learning model corresponding to each channel. The machine learning model may comprise one or more of a random forest, a boosted decision tree, a classification tree, a regression tree, a bagging tree, or a rotation forest.

Recording the plurality of EEG signals may occur over a time window. The duration of the time window may range from about 1 second to about 10 minutes. For example, the duration of the time window may be about 1 second, about 5 seconds, about 10 seconds, about 15 seconds, about 20 seconds, about 25 seconds, about 30 seconds, about 40 seconds, about 50 seconds, about 60 seconds, about 2 minutes, about 3 minutes, about 4 minutes, about 5 minutes, about 6 minutes, about 7 minutes, about 8 minutes, about 9 minutes, or about 10 minutes. In some variations, the time window includes three temporal segments. The temporal segments may or may not be continuous temporal segments. Each of the temporal segments may range from about one second to about 20 seconds. In one variation, each temporal segment is about 10 seconds.

In some variations of the method, the plurality of electroencephalography (EEG) signals is received from a plurality of electrodes arranged in a headband worn by the subject. Other methods may further include determining a seizure burden for the subject and triggering an alarm when the seizure burden is above a predetermined seizure activity threshold. Alerts (e.g. notifications) may also be used to indicate status epilepticus in the subject. Additionally, the seizure activity of the subject may be treated based on the seizure classification. For example, a medication may be administered to the subject if the EEG signals are determined to be positive for seizure.

### BRIEF DESCRIPTION OF THE DRAWINGS

FIG. 1 is a schematic illustrating an exemplary seizure detection system including a data module and a seizure detection module.

FIG. 2 is a schematic illustrating the EEG signal work flow for seizure detection in the data and seizure detection modules shown in FIG. 1.

FIG. 3 shows an exemplary seizure burden graph.

FIG. 4 is a schematic of an exemplary computer system programmed or otherwise configured to implement the methods described herein.

FIG. 5 is a schematic of an exemplary feature-based model programmed or otherwise configured to extract features from EEG signals and generate a correction factor.

FIG. 6 is a schematic of an exemplary deep learning model programmed or otherwise configured to use a neural network to classify EEG signals.

FIG. 7 is a schematic of an exemplary seizure detection system that includes the feature-based and deep learning models shown in FIGS. 5 and 6.

FIG. 8 provides seizure detection results using an exemplary hybrid model from a study of 1148 subjects across 10 hospital sites.

FIG. 9 is a schematic of an exemplary spike detection module programmed or otherwise configured to identify and characterize potential spikes from EEG signals.

FIG. 10 is a schematic of another exemplary deep learning model programmed or otherwise configured to use a neural network to classify EEG signals.

FIG. 11 is a schematic of an exemplary seizure detection system that includes the deep learning model shown in FIG. 10.

FIG. 12 is a flow chart of an of exemplary method of classifying seizures using aggregated outputs from three deep learning models.

FIG. 13 provides an exemplary annotation software interface used for data collection and annotation.

FIGS. 14A and 14B depict exemplary insights bars that may be used to highlight areas of potential concern in EEG data.

### DETAILED DESCRIPTION

This application generally relates to systems and methods for the classification and/or detection of seizure in a subject. Seizure detection may be based on the output from multiple classification models, e.g., hybrid models that may include one or more machine learning models that process a plurality of features extracted from electroencephalography (EEG) signals in combination with the output from one or more deep learning models. The output of the hybrid models may comprise, or be used to generate, a seizure burden of the subject. If seizure is detected, any suitable therapy may be given to the subject to treat the seizure (e.g., a drug, controlling the environment, and/or addressing an underlying medical condition).

Seizure is a neurological phenomenon characterized by abnormal, excessive electrical activity in the brain. It presents a range of clinical issues that may have significant implications for individuals and their overall health. Seizure occurrences may be unpredictable, disrupting daily activities and social interactions. Those living with seizures often experience apprehension about having a seizure in public or compromising their safety while engaging in tasks such as driving or operating machinery. Moreover, the consequences of seizures extend beyond the immediate event, leading to various physical and psychological complications. Physical injuries are common during seizures due to sudden loss of muscle control, resulting in falls, head injuries, and potential burns. Additionally, seizures may lead to emotional distress, anxiety, and a decreased quality of life for individuals and their families.

A lack of expert awareness and understanding of seizures among healthcare professionals may result in delayed diagnosis and treatment of seizure patients, which may have detrimental effects on patient outcomes. For example, research shows that up to one-third of newly diagnosed individuals with epilepsy in the United States remain untreated up to three years after diagnosis. To improve early detection and management, it is crucial for healthcare providers to be vigilant about the signs and symptoms of seizures. Several clinical assessment tools have been developed to aid in the identification and evaluation of seizures. These tools aim to provide a comprehensive understanding of seizure severity and associated factors. However, integrating these assessment protocols into routine clinical practice may be challenging. Passive methods that do not solely rely on behavior-based assessments administered by specialists are sought after in the ICU setting.

Recognizing the importance of prompt seizure management, healthcare organizations and professionals are actively working to improve awareness, develop effective assessment tools, and promote a patient-centered approach to care. By increasing understanding and implementing appropriate strategies, healthcare providers may better support individuals living with seizures, minimize the impact of clinical issues, and enhance overall patient outcomes. For example, seizure activity may be accurately classified using electroencephalogram (EEG) signals and predictive models as described herein. In particular, the methods and systems described herein may improve upon conventional methods and systems for seizure classification and detection by discriminating between electrographic seizures, highly pathological EEG with high likelihood of epileptiform activity, and normal electrographic activity in classifying a subject's brain activity over a period of time.

The seizure detection systems and methods described herein may provide a user with an EEG detection device comprising a data module and a seizure detection module capable of automatic, quick, and accurate seizure detection. The seizure detection module may be capable of notifying the user of an impending/active seizure. In general, the data module intakes EEG signals from an EEG device (e.g., a headband including a plurality of EEG electrodes). The EEG signals may then be processed and analyzed by the seizure detection module. During analysis, EEG features may be extracted. The EEG features may be classified using one or more feature-based machine learning models and/or one or more transform models employing neural networks. The classification of the features for a given time epoch (or window comprising multiple epochs) may then be governed by a control policy to calculate a seizure burden value. If the seizure burden value is equal to or exceeds one or more thresholds, the method and/or system may generate one or more notifications that can used by a healthcare practitioner to assess whether the subject may be at risk of having a seizure. Increasing values of seizure burden may be used by a healthcare practitioner as an indication of an increasing severity of seizures. Seizure burden values equal to or exceeding one or more thresholds may be used by a healthcare practitioner as an indication of a medical condition, for example, status epilepticus. The change in seizure burden over time, or the characteristic shape of a seizure burden graph, may be used by a healthcare practitioner to determine the course of treatment for the subject or to evaluate the effectiveness of a course of treatment for the subject.

Overall, the systems and methods for detection of seizures and seizure-like events may include an algorithm configured to stream continuous multi-channel EEG recordings, analyze EEG signals for seizure activity, including at least one of seizures, seizure-like activity, electrographic seizure, status epilepticus, highly pathological EEGs with a high likelihood of epileptiform activity, post-ictal activity, and abnormal EEG patterns. The algorithm may also be configured to calculate seizure burden, and generate alerts for seizure-like episodes and status epilepticus. The algorithm may be constructed using a mixture of multiple machine-learning models that take multi-channel, multi-segment EEG data as an input and produce outputs as mentioned above.

### Data Module

### EEG Signals/Acquisition

The system for detecting seizures may include a data module configured to receive EEG signals from one or more electrodes electrically coupled via corresponding conductive wires to a controller and/or output device. In some variations, the electrodes may be coupled to the controller and/or output device wirelessly. The electrodes may be contained within an electrode carrier system that is secured around the head of the patient. The electrode carrier system may be configured as a headband or incorporated into any number of other platforms or positioning mechanisms for maintaining the electrodes against the patient body. Individual electrode assemblies may be spaced apart from one another so that, when the headband is positioned upon the patient's head, the electrode assemblies may be aligned optimally for receiving EEG signals.

The controller and/or output device may generally comprise any number of devices for receiving the electrical signals such as electrophysiological monitoring devices and may also be used in combination with any number of brain imaging devices, e.g., fMRI, PET, NIRS, etc. In one particular variation, the electrode variations described herein may be used in combination with devices such as those which are configured to receive electrical signals from the electrodes and process them.

In some variations, signals corresponding to brain electrical activity are obtained from a human brain and correspond to electrical signals obtained from a single neuron or from a plurality of neurons. In some variations, sensors include one or more sensors affixed (e.g., taped, attached, glued) externally to a human scalp (e.g., extra-cranial sensor). For example, an extra-cranial sensor may include an electrode (e.g., electroencephalography (EEG) electrode) or a plurality of electrodes (e.g., electroencephalography (EEG) electrodes) affixed externally to the scalp (e.g., glued to the skin via conductive gel), or more generally positioned at respective positions external to the scalp Alternatively, dry electrodes can be used in some implementations (e.g., conductive sensors that are mechanically placed against a living subject's body rather than planted within the living subject's body or held in place with a conductive gel). An example of a dry electrode is a headband with one or more metallic sensors (e.g., electrodes) that is worn by the living subject during use. The signals obtained from an extra-cranial sensor may sometimes be called EEG signals or time-domain EEG signals. In some cases, a sensor may be an accelerometer or an inertial measurement unit (IMU) that may measure the mechanical movement of the subject and/or the device (e.g., produce one or more electrical signals corresponding to mechanical movement of the subject and/or device). The system may be configured to utilize one or more sensors to aid in seizure detection as described elsewhere herein.

The data module may further be configured to process acquired EEG data. In some variations, the data module may be configured to pre-process the EEG data by filtering and/or segmenting raw EEG signals into a plurality of temporal segments. For example, EEG data detected with a plurality of channels may be recorded and filtered using one or more filters (e.g., band-pass filter(s)). Next, the filtered EEG data may be segmented into a plurality of segments, which may be non-overlapping. Each temporal segment may then be analyzed and classified (independently or grouped with other temporal segments) to determine a seizure burden from the EEG signals. In some variations, one or more algorithms (e.g., a plurality thereof) working in concert may be configured to receive the series of temporal segments and produce at least one output per segment. The raw data may be recorded at a sampling rate of about 100 Hz to about 500 Hz (e.g., about 250 Hz) and filtered between about 1 Hz and about 35 Hz (e.g., using a Butterworth filter). The temporal segments may be about 1 second(s) to about 60 s, such as about 5 s to about 30 s, about 7.5 s to about 15 s, or about 10 s. Data pre-processing/processing will be described in further detail with respect to the seizure detection module, though it should be understood that, in some variations, the data module or other module (e.g., the spike detection module) may be configured to pre-process the data, and the seizure detection module may utilize the pre-processed data without having to perform further filtering or segmentation steps.

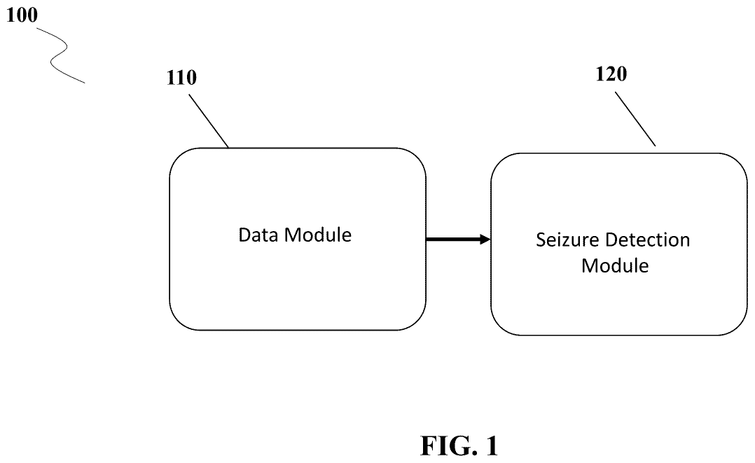

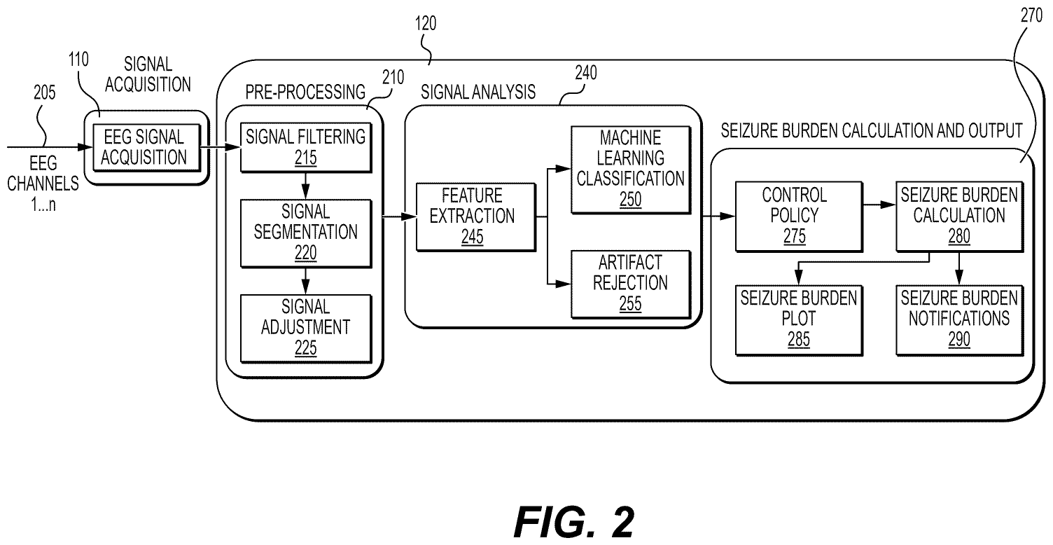

In an aspect, the present disclosure provides a method for seizure detection. In some cases, the method may include receiving a plurality of signals (e.g., EEG signals, EKG signals, EMG signals, etc.) over a plurality of channels for a subject. The method may include receiving a plurality of electroencephalography (EEG) signals over a plurality of channels for a subject. The plurality of EEG signals may be provided to a seizure detection module. FIG. 1 shows an illustration of the workflow of EEG signal collection by the EEG device module 110 to the seizure detection module 115. FIG. 2 shows an in-depth illustration of the workflow by the EEG device module and seizure detection module for seizure prediction. As shown in FIG. 2, the seizure detection module can comprise a pre-processing module, signal analysis module, and seizure burden calculation and output module. The EEG device module 110 may include a plurality of channels 205 for EEG signal acquisition from a subject. In some cases, the plurality of channels may be between 1 to 256 channels, including all values and sub-ranges therein. In some cases, the plurality of channels may be between 8 to 256 channels. In some cases, the plurality of channels may be more than 256 channels. In some cases, the plurality of channels may be 8, 10, 12, 16, 19, 20, 32, 64, 128, or 256 channels. In other cases, the EEG device may include one (1) or more channels, e.g., one (1) to 19 channels. In one case, the one or more channels includes 8 channels.

### Spike Detection Module

The spike detection module may be configured to analyze the plurality of temporal segments to identify one or more potential spikes, classify the one or more potential spikes as a physiological spike or an artifactual spike based on one or more of spike parameters, and characterize each of the plurality of temporal segments as physiological or artifactual based on one or more spike metrics. In some variations, the spike detection module may additionally be configured to characterize a severity of artifacts for artifactual segments. The spike detection module may be a component of the seizure detection module, or in some variations, may be a separate module that communicates with (e.g., transmits outputs to) the seizure detection module. In general, the spike detection module may be configured to analyze filtered EEG data prior to feature extraction by the seizure detection module. In some variations, the spike detection module may generate input-features for the feature-extraction model in the form of one or more spike metrics.

The spike detection module may comprise an analog front end configured to receive sensor EEG signals from sensors. In some variations, a separate (e.g., independent) analog front end may be provided for interfacing with each of a set of sensors. In some variations, one or more analog front ends may be provided for interfacing with a set of EEG sensors. In some variations, the spike detection module may receive EEG data from the data module. The data may be pre-processed by the data module, as described above. Alternatively, the spike detection module may similarly be configured to pre-process raw EEG signals using one or more filters (e.g., a band-pass filter) and/or by segmenting the raw signals into temporal segments (e.g., of about 10 seconds).

With the pre-processed data, the spike detection module may analyze each of a plurality of temporal segments to identify potential spikes. This analysis may comprise convolving the EEG signals in each temporal segment. The convolution operation may identify local minima in the EEG traces. These local minima in the convolution may correspond to and be identified as potential spikes. For each potential spike, the spike detection module may determine one or more spike parameters, such as at least one of amplitude, width, prominence, and polarity. Spikes exhibiting one or more non-physiological parameters may be disqualified for exhibiting non-physiological properties (e.g., being artifactual). Non-physiological parameters may be those that exceed predetermined thresholds for and/or otherwise do not meet criteria for physiological activity. For example, each spike parameter may be compared to a threshold for physiological activity, and those exceeding the threshold may be classified as non-physiological.

The spike detection module may be configured to exclude non-physiological spikes from a final set of physiological spikes of the temporal segment that are further analyzed to determine one or more spike metrics for the temporal segment. These spike metrics may include, for example, at least one of frequency, jitter, polarity, and spike count. Thus, in some variations, the spike detection module may classify each temporal segment as physiological or artifactual based on one or more spike metrics. For example, the spike detection module may compare the spike metrics for each temporal segment to predetermined thresholds to determine whether the temporal segment is artifactual. Additionally, or alternatively, a temporal segment may be classified as artifactual when an impedance associated with an electrode used to record the EEG signals exceeds a threshold.

In some variations, the spike detection module may be configured to generate the one or more spike metrics by: filtering the EEG signals in each temporal segment to identify potential spikes; determining one or more spike parameters for each potential spike; classifying each potential spike as physiological or non-physiological based on a threshold for at least one of the one or more parameters; removing the non-physiological) potential spikes from the temporal segment; and subsequently generating the one or more spike metrics for the temporal segment. The potential spikes may correspond to local minima in the EEG signals. The threshold for the at least one parameter may be channel-specific or dynamically adjusted based on a signal quality or impedance. In some variations, the threshold for the at least one parameter may be based on an acceptable range of physiological values for the parameter. The one or more parameters of each potential spike may comprise at least one of an amplitude, a width, a frequency, a prominence, and a polarity.

For physiological temporal segments, the spike metrics may be used as input features to the feature-based classifier when generating the first probability value. For artifactual segments, the spike detection module may be further configured to generate a correction factor based on the severity of the artifact. That is, the spike detection module may be configured to characterize a severity of an artifact (e.g., via a threshold comparison), generate a correction factor based on the determined severity, and may provide this correction factor downstream for combining with other outputs to determine seizure classification.

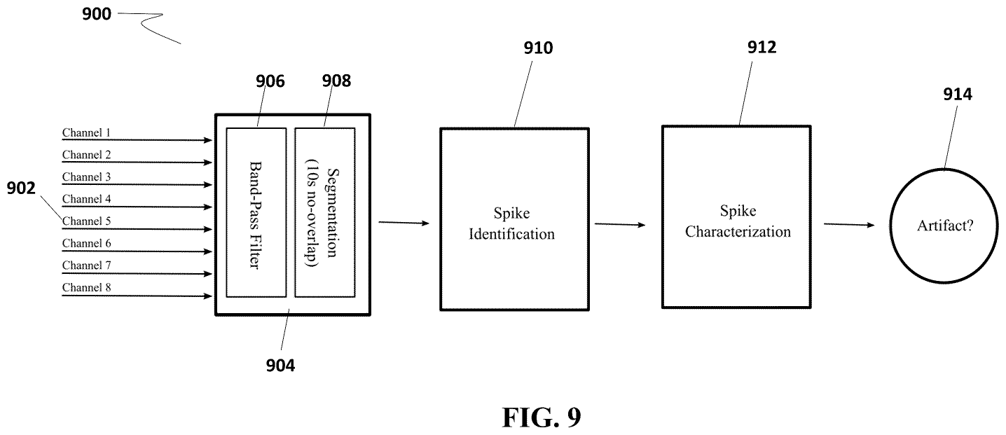

FIG. 9 is a schematic of an exemplary spike detection module programmed or otherwise configured to identify and characterize potential spikes from EEG signals. Spike detection module 900 may be configured to analyze a plurality of EEG signals received from a plurality of channels 902 (e.g., channel 1 through channel 8). The EEG signals may first be processed by a pre-processing unit 904, which may include a filtering model 906 and a segmentation model 908 configured to segment the EEG signals (e.g., into 10 second non-overlapping temporal segments). The processed EEG signals may then be passed to a spike identification model 910, which may identify potential spikes in the EEG signals. The identified potential spikes in each temporal segment may then be analyzed by a spike characterization model 912 to determine spike metrics (e.g., frequency, jitter, polarity, spike count, and/or the like). Based on the spike metrics, each temporal segment may then be classified as an artifact 914 or as a physiological temporal segment. Temporal segments identified as artifactual may be excluded from further analysis or processed with additional correction factors, while spike metrics for physiological segments maybe provided as input features to a feature-based classifier for seizure detection.

### Seizure Detection Module

The seizure detection module may generally be configured to analyze EEG signals and determine seizure classifications for individual temporal segments. This module may comprise one or more components/models, such as at least one of a pre-processing unit configured to filter and segment the EEG signals, a feature extraction model configured to derive a plurality of features from the EEG segments, an artifact rejection model to identify and address artifactual segments, a classification model configured to determine a seizure probability value for individual or groups of segments based on extracted features, a transform model configured to determine a per-channel seizure probability value, and a decision model to combine a plurality of outputs. In some variations, the classification model may comprise a plurality of different classifiers operating in parallel pathways. For example, a memoryless classifier may be applied to calculated feature-vectors to classify individual temporal segments, and a contextual classifier may be (separately) applied to a plurality of feature-vectors (i.e., a context of feature-vectors over time) to classify the plurality of temporal segments together. The outputs from these parallel processing pathways may be aggregated by the decision model that applies correction factors derived from both the feature-based model and spike detection model to determine seizure classifications for individual temporal segments. These models and functions are described in further detail below.

### (a) Pre-Processing/Processing of EEG Signals

In some variations, the seizure detection module may have one or more analog front ends configured to receive sensor EEG signals from sensors. The EEG signals may be pre-processed as described elsewhere herein. In some variations, a separate (e.g., independent) analog front end may be provided for interfacing with each of a set of sensors. In some variations, one or more analog front ends may be provided for interfacing with a set of EEG sensors.

In some variations, the method may include pre-processing the plurality of signals by segmenting the plurality of signals for each channel into a plurality of temporal data segments. In some variations, the method may include pre-processing the plurality of EEG signals by segmenting the plurality of EEG signals for each channel into a plurality of temporal data segments. FIG. 2 shows an illustration of the seizure detection module 120. The seizure detection module intakes EEG signals from a plurality of channels from the EEG device module. The seizure detection module may pre-process the EEG signals from a plurality of channels with a pre-processing module 210 configured to pre-process EEG signals. As shown in FIG. 2, the pre-processing module can include a signal filtering module 215, signal segmenting module 220, and signal adjustment module 225.

In some variations, the filtering module 215 may be configured to may filter EEG signals from the incoming set of channels from the EEG device module as described elsewhere herein. In some cases, pre-processing may be, for example, segmenting the EEG signals, filtering the EEG signals based on frequency, adjusting the EEG signals, or as described elsewhere herein, etc.

In FIG. 2, the signal segmentation module 220 can be configured to segment EEG signals. In some variations, the plurality of EEG signals may be segmented to between 1 to 100000 data segments. In some cases, the number of EEG data segments may depend on the duration of the EEG recordings. In some cases, the number of EEG data segments may be fixed regardless of the duration of the EEG recordings.

In some variations, each temporal data segment may have a duration of between about 1 second to 1 hour. In some cases, each temporal data segment may have a duration of between about 1 second to 30 seconds. In some cases, each temporal data segment may have a duration of between about 1 second to 10 seconds. In some cases, the duration of each temporal data segment may be fixed for the entire EEG recording. In some cases, the duration of each temporal data segment may be variable or adaptive during an EEG recording.

In some variations, the pre-processing of the plurality of EEG signals may comprise applying one or more filtering steps to the plurality of EEG signals over the plurality of channels. The pre-processing of the plurality of EEG signals may comprise using at least 1 filter, 2 filters, 3 filters, 4 filters, 5 filters, 6 filters, 7 filters, 8 filters, 9 filters, 10 filters, 15 filters or more. The pre-processing of the plurality of EEG signals may comprise using at most about 15 filters, 10 filters, 9 filters, 8 filters, 7 filters, 6 filters, 5 filters, 4 filters, 3 filters, 2 filters or less. The pre-processing of the plurality of EEG signals may comprise using anywhere between 1 to 15 filters, 1 to 10 filters, 1 to 5 filters, or 1 to 3 filters.

In some variations, the one or more filtering steps may be applied before, during, and/or after the segmentation of the plurality of EEG signals. One or more of the filtering steps may include, for example, a digital filter, an analogue filter, or a combination thereof. One or more of the filtering steps may include, for example, a bandpass filter, low-pass filter, a high-pass filter, a band-stop filter, an all-pass filter, a Kalman filter, an adaptive filter, or a notch filter, etc. In some cases, the low frequency cutoff of the filters may be between 0.1 Hz and 5 Hz. In some cases, the high frequency cutoff of the filters may be between 5 Hz and 200 Hz. In some cases, the notch filter frequency may match the local power line frequency. In some cases, the notch filter frequency may be 50 Hz or 60 Hz to match the local power line frequency.

In some variations, each temporal data segment may be associated with a time epoch. For each corresponding time epoch, a cluster of seizure-positive classifications may be indicative of a potential electrographic seizure. In some cases, a cluster of seizure-positive classifications may comprise of between about 1 to 50 seizure positive classifications. In some cases, a cluster of seizure positive classifications may comprise of between 1 to 10 seizure positive calculations.

In some variations, the method may further comprise comparing the classifications sequentially across a plurality of time epochs on each channel. In some cases, before/after/during comparing the classifications sequentially across a plurality of time epochs on each channel, the classifications sequentially across a plurality of time epochs on each channel may be discarded. In some cases, a subset of the classifications may be discarded. In some cases, a subset of fewer than about 1 to 20 classifications may be discarded. In some cases, a subset of fewer than 3 classifications may be discarded.

In some variations, the subset of seizure-positive classification may be discarded because, for example, they may be random readings, of low reliability, inaccurate classification, incorrect classification, calibration, system error, disconnected electrodes, artifactual signals, system interference, or other signals, etc.

In some variations, the subset of seizure-positive classification may be discarded to, for example, conserve memory space, improve processing speed, reduce energy usage, reduce heat of the system, reduce calculation costs, save processing power, save processing time, increase reliability, or decrease random access memory usage, etc.

In some variations, the greater number of seizure-positive classifications in a row may be indicative of high reliability. The greater the reliability of seizure-positive classifications, the more accurate determination of detecting a seizure in a patient. In some cases, the greater reliability of seizure-positive classifications may be indicative of the machine learning algorithm accuracy, quality of data (EEG signals), or health status of the EEG detecting system, etc.

In some variations, a particular time epoch may be classified as associated with a potential electrographic seizure if the temporal data segments for a subset of the plurality of channels are classified as seizure-positive. In some cases, the subset may be at least 5%, 10%, 20%, 30%, 40%, 50% or more of the plurality of channels. In some cases, the subset may be at most about 50%, 40%, 30%, 20%, 10%, 5%, or less of the plurality of channels.

### (b) EEG Signal Adjustment

FIG. 2 shows a signal adjustment module 225 configured to adjust an EEG signal. In some variations, the method may adjust any EEG signal. Adjusting an EEG signal may include, for example, increasing and/or decreasing the amplitude of the EEG signal, adding or decreasing the noise level of the EEG signal, increasing and/or decreasing the time epoch the EEG signal, increasing and/or decreasing the intensity of the EEG signal, increasing and/or decreasing the signal frequency of the EEG signal, increasing and/or decreasing the voltage of the EEG signal, changing the morphology of the EEG signal (e.g., the shape of the EEG signal), increasing and/or decreasing the periodicity of the EEG signal, increasing or decreasing the synchrony of the EEG wave, spectral subtraction, standardizing etc.

In some cases, the EEG signal may be reduced. In some cases, the EEG signal may be down-sampled to a lower sampling frequency. For example, EEG data recorded at a sampling frequency of 500 Hz may be down sampled by a factor of 2 to 250 Hz.

In some cases, the EEG signal may be subjected to bit-width reduction. In some cases, the level of resolution at which the EEG signals are recorded may not be required by the method to achieve accurate seizure detection. In some cases, the bit-width reduction may reduce the EEG signal to a lower number of bits per sample through standard quantization of the EEG signal, for example, from 32 bits per sample to 12 bits per sample. In some cases, bit-width reduction may be advantageous if the method is to be implemented in a portable system, as it may be useful for reducing power consumption due to decreased processing load.

In some cases, spectral subtraction may be used to reduce the amount of additive noise in the EEG signal. In some cases, the noise may be caused by external surroundings. In some cases, the noise may be caused by the measurement equipment. In some cases, the noise may be caused by the user. In some cases, an average frequency spectrum of non-seizure EEG signal may be computed over a period of time to provide a base level estimate of the noise frequency spectrum. In some cases, as the EEG signals are recorded, the EEG signals may be converted to the frequency domain. In some cases, the average noise spectrum may then be subtracted from the EEG frequency spectrum. In some cases, the resulting spectrum and phase information from the original noisy signal may be combined. In some cases, the resulting spectrum may be transformed back into time domain to produce a de-noised signal.

In some variations, the EEG signal may be standardized by eliminating the effect of the montage that was used in gathering the EEG signals. In some cases, independent component analysis (ICA) or principal component analysis (PCA) methods may be used to provide the montage elimination. In some cases, the ICA or PCA method may separate the EEG signal into a set of sources independent of the montage used to record them. In some cases, using standardized EEG data may remove errors introduced by the varying practices of clinicians.

In some cases, a non-negative matrix factorization (NMF) method may be applied to each channel as a form of artifact removal. In some cases, the spectrum of the signal may be decomposed into the extracted bases to obtain weights. In some cases, the spectrum may be reconstructed using the bases of artifacts and the corresponding weights removed from the initial EEG signal.

### (c) Signal Analysis Using Feature Extraction

In FIG. 2, the seizure detection module may comprise a signal analysis module. The signal analysis module may comprise a feature extraction module 245 and a machine learning classification module 250. The feature extraction module 245 may be configured to take pre-processed measured data (e.g., EEG signals) from the pre-processing module 210 to build derived values (e.g., features). In some variations, feature extraction may start from an initial set of measured data (e.g., EEG signals, EEG signals of a given time epoch, etc.) and may build derived values (e.g., features) intended to be informative and non-redundant. In some variations, the feature extraction model may receive one or more spike metrics for the temporal segment (from the spike detection module) as input-features. In some cases, the feature extraction module may include extracting a plurality of features from each temporal data segment for each channel individually. In some cases, the feature extraction module may include extracting a plurality of features from each temporal data segment for all channels together. In some cases, the feature extraction module may include extracting a plurality of features from each temporal data segment of one or more groupings with each grouping consisting of one or more channels. As shown in FIG. 2, the extracted features can be relayed to a machine learning classification module 250 that may be configured to analyze and classify the extracted features as described elsewhere herein. In some cases, feature extraction may facilitate the subsequent learning and generalization steps of a machine learning algorithm. In some cases, feature extraction may lead to better human interpretations. In some cases, feature extraction may be related to dimensionality reduction.

In some cases, the features may include a plurality of discrete values associated with the time domain, frequency domain, time-frequency domain, information theory, and nonlinear-dynamics system theory features. In some cases, the plurality of features may include a plurality of discrete values associated with the time and/or frequency domain features. The plurality of features may include a plurality of continuous values associated with the time and/or frequency domain features.

The features may be computed using time-frequency analysis of data recorded on individual channels. In some variations, one or more types of features may be computed for each channel, including temporal properties, regularity features, power spectrum measurements, synchrony measures, and self-similarity measures. Temporal properties may include signal characteristics measured in the time domain, such as signal range, zero crossings, variance, or similar metrics. Regularity features may be computed to quantify the information content of the signal, for example, using entropy-based metrics. Power spectrum measurements may include spectral components of the signal, such as Alpha, Beta, Delta, or Theta frequency bands. Synchrony measures may be computed to determine the extent of coherence between signals from different brain regions, for instance, using delta phase correlation. Self-similarity measures may be used to evaluate similarity within the signal across time and space, such as based on auto-correlogram properties.

In some cases, when the input data (e.g., EEG signals) to the machine learning algorithm is too large to be processed and suspected to be redundant (e.g., the same measurement in both Hz and seconds, or the repetitiveness of a characteristic), the data can be transformed into a reduced set of features.

In some cases, determining a subset of the initial features may be called feature selection. In some cases, the selected features may be expected to contain the relevant information from the input data (e.g., EEG signals). In some cases, the selected features may be expected to contain the relevant information from the input data so that the desired task can be performed by using this reduced representation instead of the complete initial data.

In some variations, feature extraction may involve reducing the number of resources required to describe a large set of data (e.g., EEG signals). In some cases, analysis with a large number of variables may require a large amount of memory and computation power. In some cases, it may cause a machine learning algorithm to overfit to training samples and generalize poorly to new samples. In some cases, feature extraction may construct combinations of the variables to accurately describe the data with sufficient accuracy. In some cases, feature extraction may construct combinations of the variables to accurately describe the data with sufficient accuracy while preventing overfitting.

In some variations, results may be improved using constructed sets of application-dependent features. In some cases, the constructed sets may be built by an expert. In some cases, general dimensionality reduction techniques may be used. In some cases, general dimensionality reduction techniques may be, for example, independent component analysis, isomap, kernel PCA, latent semantic analysis, partial least squares, principal component analysis, multifactor dimensionality reduction, nonlinear dimensionality reduction, multilinear principal component analysis, multilinear subspace learning, semidefinite embedding, autoencoder, etc.

In some cases, a set of numeric features may be described by a feature vector. In some cases, a feature vector may be an n-dimensional vector of numerical features that represent some object.

In some variations, data analysis software packages may provide for feature extraction. In some cases, data analysis software packages may provide for dimension reduction. In some cases, data analysis software packages may include programming environments such as MATLAB, SciLab, NumPy, or the R language, etc. In some cases, a programming language script may be used to extract features from EEG signals. In some cases, the programming language script may be, for example, MATLAB, python, java, javascript, Ruby, C, C++, or Perl, etc.

In some cases, the plurality of features may be intrinsic in the plurality of EEG signals. Intrinsic may be a feature of an EEG signal that may be measured, for example, the amplitude of the EEG signal, the duration of the EEG signal, the variation of the EEG signal, the power of the EEG signal, the local maxima/minima of the EEG signal, the pattern of the EEG signal, the regularity of the EEG signal, the spectral power distribution of the EEG signal, or the frequency of the EEG signal, etc. In some cases, the plurality of features may be a measurement of the power of a signal with a particular frequency. The frequency may be, for example, from about 0 Hz to 100 Hz. In some cases, the power of a signal may be normalized to a total power. In some cases, the power of a signal may be a ratio of power between one or more signals. In some cases, a feature may be a function performed on a signal to obtain a value. For example, a function may measure the root mean square (RMS) of a signal (e.g., EEG signal) to obtain the RMS value of the signal. In some cases, a feature may compare one signal (e.g., EEG signal) to one or more signals. In some cases, a feature may compare one or more signals (e.g., EEG signals) to one or more signals. In some cases, a feature may measure an attribute of a signal (e.g., EEG signal). In some cases, a feature may compare one or more attributes of a signal (e.g., EEG signal) with one or more attributes of a signal. An attribute may be, for example, an intrinsic property of the EEG signal. In some case, the feature of an EEG signal may be continuous and/or discrete in time.

In some cases, the plurality of features may include at least twenty different time and/or frequency features. In some cases, the plurality of features may include at most one thousand time and/or frequency features. In some cases, the plurality of features may include between about 10 features to 200 features. In some cases, the plurality of features may include between about 10 features to 100 features. In some cases, the plurality of features may include between about 10 features to 50 features.

In some cases, the plurality of signals may be converted into a digital signal. In some cases, the plurality of signals may be converted into a digital signal and then an analog signal.

In some cases, the features may be sampled from a portion of the EEG signal. Features may be sampled from a portion of the EEG signal to reduce processing time and power required.

In some variations, a feature may be pertaining to a certain weight value. The weight value may give one feature a higher score for detecting a seizure. The higher score may indicate that the feature may be more relevant in predicting seizure activity. The method may adjust the weight value of any feature at any given time. The method may adjust by increasing and/or decreasing the weight value of any feature at any given time.

### (d) Artifact Rejection

In some variations, one or more of the features may be used with machine learning or conventional statistical techniques to determine if a segment is likely to contain artifacts. FIG. 2 shows the artifact rejection module 255 which identifies segments containing artifacts. In some variations, the artifact rejection module may receive outputs from the spike detection module (e.g., correction factors and/or spike metrics) to aid in identifying segments containing artifacts. The identified artifacts may be a result of electrical interference, electrode instability or movement, subject movement, subject eye movement or blinking, subject chewing, subject muscle tensing, subject electrocardiogramartifact, etc. In some cases, movement sensors or other sensors may be used as an additional input to the artifact rejection module. In some cases, the identified artifacts can be rejected from being used in seizure classification. In some cases, the identified artifacts can be reduced, cancelled, or eliminated and the remaining signal may still be processed for seizure classification. In some cases, the machine learning algorithm may prioritize certain features to reduce calculation costs, save processing power, save processing time, increase reliability, or decrease random access memory usage, etc.

Additionally, or alternatively, the artifact rejection model may be configured to mark artifacts using impedance data (e.g., most-recent electrode impedance measurement(s)). For example, one or more impedance measurements from an electrode that recorded a given segment may be compared to a predetermined threshold, and when the impedance measurement(s) surpass the threshold, the segment may be determined to be artifactual. Thus, the artifactual segment may not be used for subsequent analysis/predictions.

### (e) Classification Using Machine Learning

The seizure detection module may utilize one or more classification models to analyze and categorize EEG signal segments. As used herein, the terms “classification model” and “classifier” are used interchangeably throughout. The classification model may receive inputs from the pre-processing unit and the feature extraction model, with additional input features potentially derived from the spike detection module for physiological segments. The classification model may include at least one of a memoryless classifier and a contextual classifier. In some variations, seizure detection module may run a plurality of classifiers, such as first and second classifiers (e.g., memoryless and contextual classifiers), in parallel. The memoryless classifier may be configured to evaluate each temporal segment independently using machine learning algorithms (such as random forest models) to process extracted features to generate seizure probability values. The contextual classifier may be configured to analyze relationships between adjacent temporal segments using machine learning algorithms (e.g., recurrent neural networks) to identify patterns that evolve over time, thereby accounting for the temporal context of EEG signals. Each classifier may generate probability outputs and associated correction factors for the processed segments, which may be aggregated and combined with any applicable correction factors by the decision model to determine seizure classifications.

In some variations, the method may include applying a machine learning algorithm to the plurality of features to perform a seizure classification for each temporal data segment for each channel individually. In some cases, the machine learning classification module may include performing seizure classification for each temporal data segment for all channels together. In some cases, the machine learning classification module may include performing seizure classification for each temporal data segment of one or more groupings with each grouping consisting of one or more channels. FIG. 2 shows the machine learning classification module 250 that may take the features collected/extracted from the pre-processing step and classify the features. In some cases, the features may be extracted without a pre-processing step.

In some cases, machine learning algorithms may need to extract and draw relationships between features as conventional statistical techniques may not be sufficient. In some cases, machine learning algorithms may be used in conjunction with conventional statistical techniques. In some cases, conventional statistical techniques may provide the machine learning algorithm with pre-processed features.

In some variations, the plurality of features may be classified into any number of categories. A temporal segment may be classified as, for example, seizure-positive, seizure-negative, seizure-like, post-ictal, uncertain seizure activity, etc. In some cases, the plurality of features may be classified into between 1 to 20 categories. Individual categories may also be divided into sub-categories. For example, a temporal segment classified as seizure-positive may be further sub-divided into focal versus generalized seizure events.

In some variations, the method may include applying a machine learning algorithm to the plurality of features to perform a seizure binary classification for each temporal data segment for each channel.

In some variations, the one or more features collected may be discarded prior to or during machine learning classification.

In some variations, a human may select, and discard features prior/during machine learning classification. In some cases, a computer may select and discard features. In some cases, the features may be discarded based on a threshold value.

In some variations, any number of features may be classified by the machine learning algorithm. The machine learning algorithm may classify at least 10 features. In some cases, the plurality of features may include between about 10 features to 200 features. In some cases, the plurality of features may include between about 10 features to 100 features. In some cases, the plurality of features may include between about 10 features to 50 features In some variations, the machine learning algorithm may be, for example, an unsupervised learning algorithm, supervised learning algorithm, or a combination thereof. The unsupervised learning algorithm may be, for example, clustering, hierarchical clustering, k-means, mixture models, DBSCAN, OPTICS algorithm, anomaly detection, local outlier factor, neural networks, autoencoders, deep belief nets, hebbian learning, generative adversarial networks, self-organizing map, expectation-maximization algorithm (EM), method of moments, blind signal separation techniques, principal component analysis, independent component analysis, non-negative matrix factorization, singular value decomposition, or a combination thereof. The supervised learning algorithm may be, for example, support vector machines, linear regression, logistic regression, linear discriminant analysis, decision trees, k-nearest neighbor algorithm, neural networks, similarity learning, or a combination thereof. In some variations, the machine learning algorithm may comprise a deep neural network (DNN). The deep neural network may comprise a convolutional neural network (CNN). The CNN may be, for example, U-Net, ImageNet, LeNet-5, AlexNet, ZFNet, GoogleNet, VGGNet, ResNet18 or ResNet, etc. Other neural networks may be, for example, deep feed forward neural network, recurrent neural network, LSTM (Long Short Term Memory), GRU (Gated Recurrent Unit), Auto Encoder, variational autoencoder, adversarial autoencoder, denoising auto encoder, sparse auto encoder, boltzmann machine, RBM (Restricted BM), deep belief network, generative adversarial network (GAN), deep residual network, capsule network, or attention/transformer networks, etc.

In some variations, the machine learning algorithm may be, for example, a random forest, a boosted decision tree, a classification tree, a regression tree, a bagging tree, a neural network, or a rotation forest. The machine learning algorithm may be individually applied to the plurality of features extracted for each channel, such that each channel may have a separate iteration of the machine learning algorithm.

In some variations, the method may apply one or more machine learning algorithms. In some variations, the method may apply one or more one machine learning algorithms per channel.

In some variations, correction factors may be generated based on probabilities derived from the classifier to refine seizure detection accuracy. The feature-based classifier may generate a first probability value for each of the temporal segments derived from the plurality of extracted features. Based on this first probability value, a correction factor may be generated. In some variations, the correction factor may be generated when the first probability value is below a predetermined threshold, which may range from, for example, about 0.40 to about 0.70, including all values and sub-ranges therein. As previously mentioned, the predetermined threshold range may be about 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, or 0.70. The generation of the correction factor may involve a mathematical transformation of the first probability value, such as a scaling operation, or may be determined using a look-up table that maps probability ranges to corresponding correction values.

The correction factor may be applied to adjust a probability value generated by the transform model. In some variations, the correction factor may be subtracted from this probability value. This subtraction operation may be particularly useful for reducing false positives in situations where the feature-based model indicates a low likelihood of seizure activity while the transform model indicates a higher likelihood. This output combination is described in further detail herein.

In FIG. 2, the machine learning classification module 250 may comprise any number of machine learning algorithms. In some variations, the random forest machine learning algorithm may be an ensemble of bagged decision trees. In some cases, the ensemble of bagged decision trees may classify each temporal data segment for each channel as (1) seizure-positive or (2) seizure-negative. The ensemble may be at least about 1, 2, 3, 4, 5, 10, 20, 30, 40, 50, 60, 70, 80, 90, 100, 120, 140, 160, 180, 200, 250, 500, 1000 or more bagged decision trees. The ensemble may be at most about 1000, 500, 250, 200, 180, 160, 140, 120, 100, 90, 80, 70, 60, 50, 40, 30, 20, 10, 5, 4, 3, 2 or less bagged decision trees. The ensemble may be from about 1 to 1000, 1 to 500, 1 to 200, 1 to 100, or 1 to 10 bagged decision trees.

In some variations, the method may include applying a machine learning classifier to any number of channels. The method may include applying a machine learning classifier to at least about 1, 2, 3, 4, 5, 6, 7, 8, 9, 10, 15, 20, 25, 50, 100, 500, 1000 or more channels. The method may include applying a machine learning classifier to at most about 1000, 500, 100, 50, 25, 20, 15, 10, 9, 8, 7, 6, 5, 4, 3, 2 or less channels. The method may include applying a machine learning classifier from about 1 to 1000, 1 to 100, 1 to 25, or 1 to 5 channels.

In some variations, the method may include applying a machine learning classifier to a subset of channels. The subset of channels may be at least about 1%, 5%, 10%, 20%, 30%, 40%, 50% or more of the total set of channels. The subset of channels may be at most about 50%, 40%, 30%, 20%, 10%, 5%, 1% or less of the total set of channels. The subset of channels may be from about 1% to 50%, 1% to 40%, 1% to 30%, 1% to 20%, 1% to 10%, or 1% to 5% of the total set of channels.

In some variations, the machine learning algorithm may have a variety of parameters. The variety of parameters may be, for example, learning rate, minibatch size, number of epochs to train for, momentum, learning weight decay, or neural network layers etc. In some variations, the learning rate may be between about 0.00001 to 0.1. In some variations, the minibatch size may be at between about 16 to 128. In some variations, the neural network may comprise neural network layers. The neural network may have at least about 2 to 1000 or more neural network layers.

In some variations, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some variations, the momentum may be at least about 0.1, 0.2, 0.3, 0.4, 0.5, 0.6, 0.7, 0.8, 0.9 or more. In some variations, the momentum may be at most about 0.9, 0.8, 0.7, 0.6, 0.5, 0.4, 0.3, 0.2, 0.1, or less.

In some variations, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more.

In some variations, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some variations, the machine learning algorithm may use a loss function. The loss function may be, for example, regression losses, mean absolute error, mean bias error, hinge loss, Adam optimizer and/or cross entropy.

In some variations, the parameters of the machine learning algorithm may be adjusted with the aid of a human and/or computer system.

In some variations, the machine learning algorithm may prioritize certain features. The machine learning algorithm may prioritize features that may be more relevant for detecting seizures. The feature may be more relevant for detecting seizures if the feature is classified more often than another feature. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

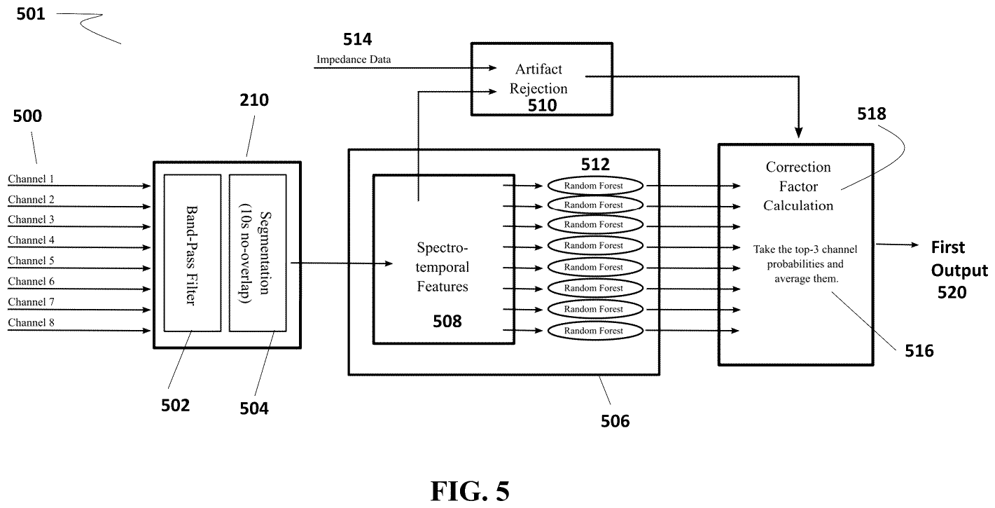

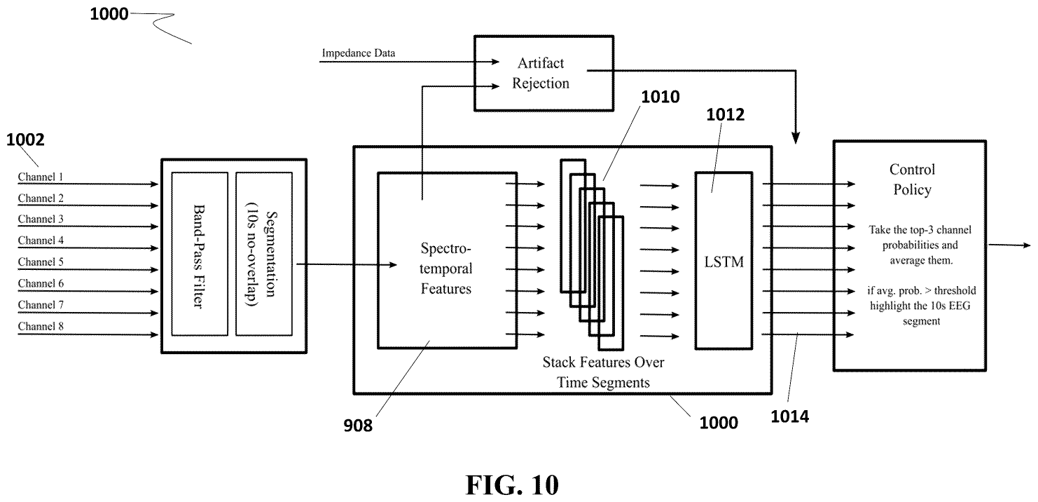

As noted above, the classification model may comprise one or both of a memoryless classifier and a contextual classifier, both of which will now be described with reference to FIG. 5 and FIG. 10, respectively.

### Memoryless Classification

The seizure detection module may comprise a memoryless classification model configured to classify each of a plurality of temporal segments independently using an extracted plurality of features, and without considering contextual information from adjacent segments. In some variations, the memoryless classifier may employ a random forest algorithm wherein each channel of the plurality of channels is assigned to an independent machine learning model instance. For each channel, the extracted plurality of features may be applied to the machine learning model corresponding to each channel. A suite of random forests (e.g., 1-per EEG channel) may be applied to the calculated feature vectors to classify them into seizure-positive versus seizure-negative classes as a probability measure.

FIG. 5 is a schematic of an exemplary process pathway 501 executed by a feature-based model 506, which may comprise a feature extraction model 508 and memoryless classifier 512. The process 501 may first include obtaining the EEG signals from the subject. In some variations, a data module 110 receives EEG signals from a plurality of electrodes arranged in a headband worn by the subject. The data module 110 may be configured to receive and record the plurality of EEG signals over one or a plurality of channels. The plurality of channels may range between 2 and 19 channels. For example, the plurality of channels may be 2, 3, 4, 5, 6, 7, 8, 9, 10, 11, 12, 13, 14, 15, 16, 17, 18, or 19 channels. In some variations, the plurality of channels may include 8 channels. The multi-channel EEG signal data may then be transmitted to the seizure detection module for pre-processing, e.g., filtering, segmentation, and/or signal adjustment, as previously described and as shown in FIG. 2. In some variations, as shown in FIG. 5, 8-channel EEG data 500 may be generated from raw waveforms recorded at a sampling rate of 250 Hz may be band-pass filtered 502 between 1 and 35 Hz using a 5th order Butterworth filter. Next, the filtered EEG data may be segmented 504 into 10 second non-overlapping time windows. The overall algorithm may take in a series of EEG segments and produce a series of outputs, one per EEG segment.

After filtering and segmentation, the EEG segments may be processed by the feature-based classifier 506. Prior to feature extraction, the EEG signals may be detrended (e.g., adjusted) so that the EEG segments have a 0-mean. After detrending the signal, the feature-based model 506 may perform feature extraction using the feature extraction model 508, artifact calculation using an artifact rejection model 510, and classification using the memoryless classifier 512. When feature extraction is performed, a 10 second window may be employed, and for each 10 second window, feature vectors may be computed using time-frequency analysis of data recorded on individual channels. The memoryless classifier 512 may comprise random forests configured to be applied to the calculated feature vectors to classify them into seizure-positive versus seizure-negative classes as a probability measure. A control policy 516 may be applied to these probabilities to obtain an average probability. The average probability may be calculated by taking the average of the top two, three, or four channel probabilities.

The artifact rejection model 510 may identify and remove artifacts from the EEG signal. For example, as described above, an EEG window may be marked as an artifact if a predefined set of features cross certain threshold values. Further, if the most recently reported impedance (e.g., impedance data 514) on an electrode is higher than a preset threshold, the segment may also be marked as an artifact. When an EEG window is marked as an artifact, it is generally not used for subsequent analysis/predictions. Instead, a correction factor may be calculated based on the severity of the artifact and applied to the output of the transform model. Classification of the temporal segments may also be used to generate a correction factor 518, which may then be used to correct the output of the transform model (e.g., the second output), as further described below.

The random forest models may be trained on annotated EEG data wherein each model evaluates a temporal segment independently during training without consideration of adjacent segments. This memoryless training approach may allow the model to focus on discriminative features present within individual segments rather than relying on temporal patterns.

### Contextual Classification

FIG. 10 is a schematic of an exemplary feature-based model 1000, which may comprise a feature extraction model 1008 and a contextual classifier 1012.

As shown in FIG. 10, the contextual classification model 1000 may receive pre-processed EEG data from a plurality of channels 1002 (e.g., channel 1 through channel 8). The pre-processed EEG data may then be provided to feature extraction model 1008, which may extract a plurality of features from the EEG signals. The extracted features may be similar to those used in the memoryless classification, including time-domain, frequency-domain, and time-frequency domain features.

The contextual classifier 1012 may differ from a memoryless classifier (e.g., memoryless classifier 512) in how it processes the extracted features. Rather than applying the features directly to independent channel classifiers, the contextual classifier 1012 may be configured to stack features over time segments 1010. This stacking operation may create a temporal sequence of feature vectors that spans a plurality of consecutive EEG segments. In some variations, the context window may span about 3 to about 30 temporal segments (including all values and sub-ranges therein), allowing the model to analyze patterns that develop over periods ranging from about 30 seconds to about 5 minutes. For example, the context window may include about 3 temporal segments, about 4 temporal segments, about 5 temporal segments, about 6 temporal segments, about 7 temporal segments, about 8 temporal segments, about 9 temporal segments, about 10 temporal segments, about 11 temporal segments, about 12 temporal segments, about 13 temporal segments, about 14 temporal segments, about 15 temporal segments, about 16 temporal segments, about 17 temporal segments, about 18 temporal segments, about 19 temporal segments, about 20 temporal segments, about 21 temporal segments, about 22 temporal segments, about 23 temporal segments, about 24 temporal segments, about 25 temporal segments, about 26 temporal segments, about 27 temporal segments, about 28 temporal segments, about 29 temporal segments, or about 30 temporal segments.

The contextual classifier 1012 may comprise a recurrent neural network, such as a Long Short-Term Memory (LSTM) network. The LSTM network may be configured to process sequential data and capture long-term dependencies. The LSTM may analyze the sequence of feature vectors to identify patterns that evolve over time, thereby accounting for the temporal context of EEG signals. This approach may be particularly effective for detecting seizure patterns that develop gradually or exhibit characteristic temporal evolutions that would not be apparent when analyzing individual segments in isolation.

Outputs from the contextual classifier 1012 may be combined (e.g., by a decision model) to generate a set of aggregate channel outputs 1014. In some variations, the contextual classifier 1012 may evaluate a plurality of outputs using different context windows, and these outputs may be condensed into a single value by taking one or more of a max, mean, or median of the contextual classifications.

The feature-based model 1000 may be trained using context-dependent training wherein weighted combinations from different patients and time-windows are pooled together to force the model to learn long-term patterns over individual patients and time. This training approach may enable the model to recognize seizure patterns that evolve over longer durations and to distinguish these patterns from non-seizure activities.

### (f) Transformation Using Machine Learning

In some variations, the seizure detection module may employ a transform model to process EEG signals for seizure detection. The transform model may provide a parallel computational pathway that processes pre-processed EEG signals to generate seizure probability values. The transform model may differ from feature-based approaches in that it may operate directly on the pre-processed EEG data rather than on extracted features, facilitating identification of complex patterns that may not be captured by predefined feature sets. The transform model may generally comprise an encoder and a decoder. The encoder may be configured in a first step to ingest the raw EEG data from multiple EEG channels at once and learn complex interactions over space and time simultaneously. Encoding EEG data in this manner may be beneficial given that the encoder may learn an encoding best suited for differentiating (e.g., classifying) seizure-like EEG from non-seizure EEG. The decoder may be configured in a second step to learn how these encodings may evolve over time. For example, a feedforward neural network and/or recurrent neural network may be used to decode the sequence of encodings.

In some variations, the transform model may incorporate attention mechanisms within both the encoder and decoder components. These attention mechanisms may allow the model to focus on the most relevant portions of the input data when making predictions. The attention mechanisms may be implemented as self-attention layers, cross-attention layers, or a combination thereof. The attention weights may be computed based on various similarity metrics, such as dot-product attention, scaled dot-product attention, or additive attention.

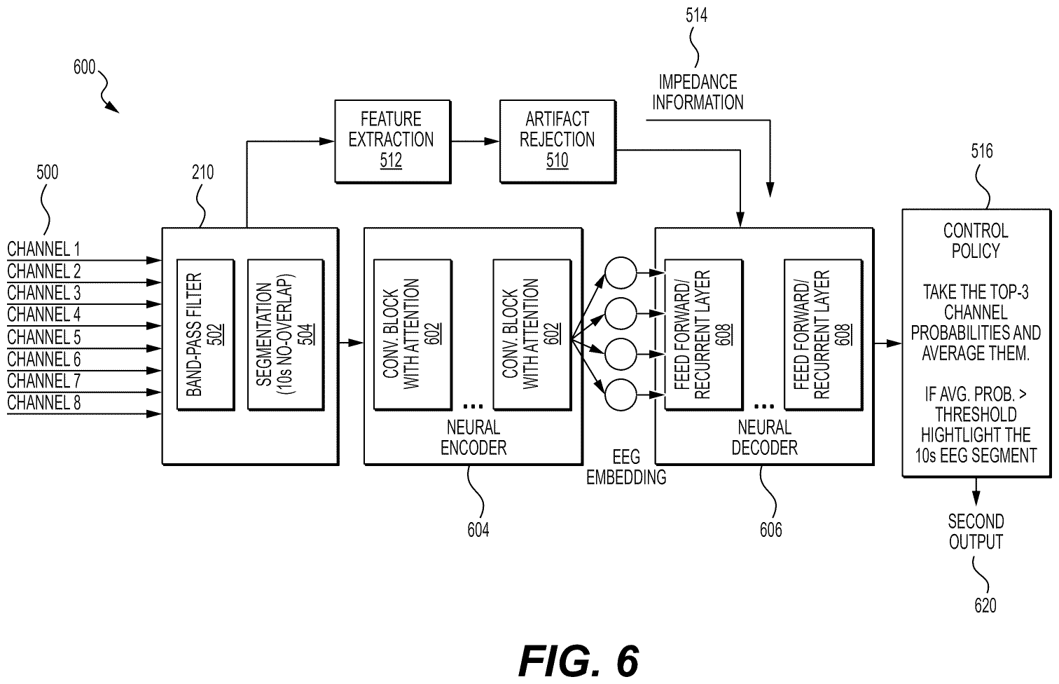

FIG. 6 is a schematic of an exemplary transform model 600, which runs a processing pathway utilizing an encoder 604 and a decoder 606. The encoder 604 may be configured to transform the pre-processed multi-channel EEG data into a plurality of vectors. In some variations, the encoder 604 may include one or more convolutional neural networks with attention blocks 602. The convolutional blocks 602 may contain a plurality of subunits (e.g., 2, 3, 4, 5, or more than 5 subunits) that perform time-compression (e.g., reduce the time samples by, e.g., 50%), space-compression (e.g., reduce the spatial-dimension by e.g., 50%), and self-attention. Alternative compression rates may be employed, such as time sample reduction by 25% to 75% and spatial-dimension reduction by 25% to 75%. These operations may be performed by using 1-D convolution operations, although 2-D convolution operations may be employed in some variations. For the spatial dimension, circular boundary conditions may be used, although other boundary conditions such as zero-padding, reflection, or replication may be employed in alternative variations.

The neural encoder 604 may translate raw multi-channel, multi-segment EEG into a multi-channel, multi-segment EEG encoding where each segment may be encoded as a separate feature vector. The dimensionality of these feature vectors may vary based on the specific implementation. The encoder 604 may be configured to transform each temporal segment into a feature vector and stack the feature vectors to generate a sequence input for further processing. This stacking operation may create a temporal sequence spanning multiple consecutive EEG segments, allowing the model to analyze patterns that develop over extended periods.

The feature vectors generated by the encoder 604 may then be stacked in time and fed into a neural decoder 606, which may produce a sequence of multi-channel outputs, each corresponding to a seizure probability of the corresponding channel. The neural network decoder 606 may include a mixture of Feed-Forward and Recurrent units 608 (e.g., GRU, LSTM, etc.), to classify a variable length series (e.g., up to 10 minutes, although shorter or longer durations such as 1 minute to 30 minutes may be employed) of EEG data into a series of continuous valued predictions. This architecture may allow the model to capture a long duration evolution of brain activity, which may facilitate detecting seizure patterns that develop gradually over time. In some variations, one or more features used in the feature-based classification may be reused to adjust the output of the decoder 606. This feature reuse may create a hybrid approach where the transform model benefits from both its direct processing of raw EEG data and the structured information captured by the feature extraction process.

The output from the transform model 600 may be a probability value indicating the likelihood of seizure activity for each temporal segment. This probability value may be combined with the probability value from the feature-based model and/or any correction factors to determine the final seizure classification. The aggregation logic, as described in relation to the control policy, may be applied to the probabilities over time to generate a raw output from the neural-network model. Each corresponding channel may receive a seizure probability value, and these channel-specific probabilities may be further processed according to the control policy.

In some variations, if the feature-based model generates a low first probability value for seizure on an EEG segment, a correction factor may be subtracted from the probability value generated by the transform model. If, after this correction, the transform model's output remains above a predetermined threshold (e.g., 0.40 to about 0.70), the EEG segment may be classified as seizure-positive.

Further, a control policy may be applied to the probabilities generated by the transform model over time to generate a raw output. In the same manner as described for the classifiers herein, the control policy may be determined by calculating the average of the top three probabilities (or alternatively, the top two, top four, or top N, where N may range from 1 to the total number of channels) for temporal segments being seizure-positive. The transform model 600 may operate in parallel with other classification models.

The transform model may be trained using a variety of optimization algorithms, such as stochastic gradient descent (SGD), Adam, RMSprop, or AdaGrad. The learning rate may range from 1e-5 to 1e-2, with learning rate schedules such as step decay, exponential decay, or cosine annealing potentially being employed to improve convergence. The transform model may be pre-trained using existing data from other subjects but may be capable of adapting to work on new subjects without extensive retraining. This adaptability may be achieved through transfer learning techniques, wherein most of the model parameters are preserved from the pre-training phase while a subset of parameters, typically in the later layers of the network, are fine-tuned for the new subject. This approach may allow the model to leverage general patterns learned from a large dataset while adapting to the specific characteristics of an individual subject's EEG patterns.

Although the classifiers for the transform model have been described as encoders and decoders, it should be understood that the transform model may additionally or alternatively employ any suitable classifier.

### (g) Signal Analysis-Combining Feature Extraction/Classification and Deep Learning

In some variations, the systems and methods described herein may include multiple models to form a hybrid model. The hybrid model may combine feature-based and deep learning models to detect various types of seizure activity. The combination may increase the accuracy (e.g., by removing false positives) and speed of seizure detection. The feature-based models (e.g., feature-based classifiers) may employ machine learning to improve the quality of the EEG signal by deriving features that focus on a distinct characteristic of an EEG signal, allowing the machine learning algorithm to more easily discern measured EEG signals and classify them. Any of the machine learning models described herein may be used (e.g., in any combination). The deep learning models (e.g., deep learning classifiers) may include the use of one or more neural networks, e.g., a convolutional neural network (CNN), a deep convolutional neural network, and a recurrent neural network (RNN), to analyze and classify the EEG signals for seizure detection. Other suitable neural networks or predictive models may also be employed (e.g., one or more of the specialized models described herein). A control policy and seizure burden calculation may be implemented after classification of the EEG signals to further help improve the accuracy of seizure detection.

In some variations, at least some of the deep learning methods may run in parallel with one or more feature-based models in a transform model. Each feature-based model and transform model may be coupled with a corresponding decision model configured to determine the top channel outputs (e.g., top 1, 2, 3, 4, or more than 4 probabilities) from each model. Each decision module may further configured to combine these outputs together with any correction factors (e.g., from the artifact rejection model and/or spike detection module). The top-outputs of the transform model may then be combined with the top-outputs from each of one or more feature-based classification models (e.g., from both a memoryless classifier and a contextual classifier) when comparing to the control policy decision framework (described in detail below). The use of both extracted features obtained as a first output from one or more feature-based models, and a data-driven, second output from a transform model may increase the seizure detection accuracy and processing speed of the system.

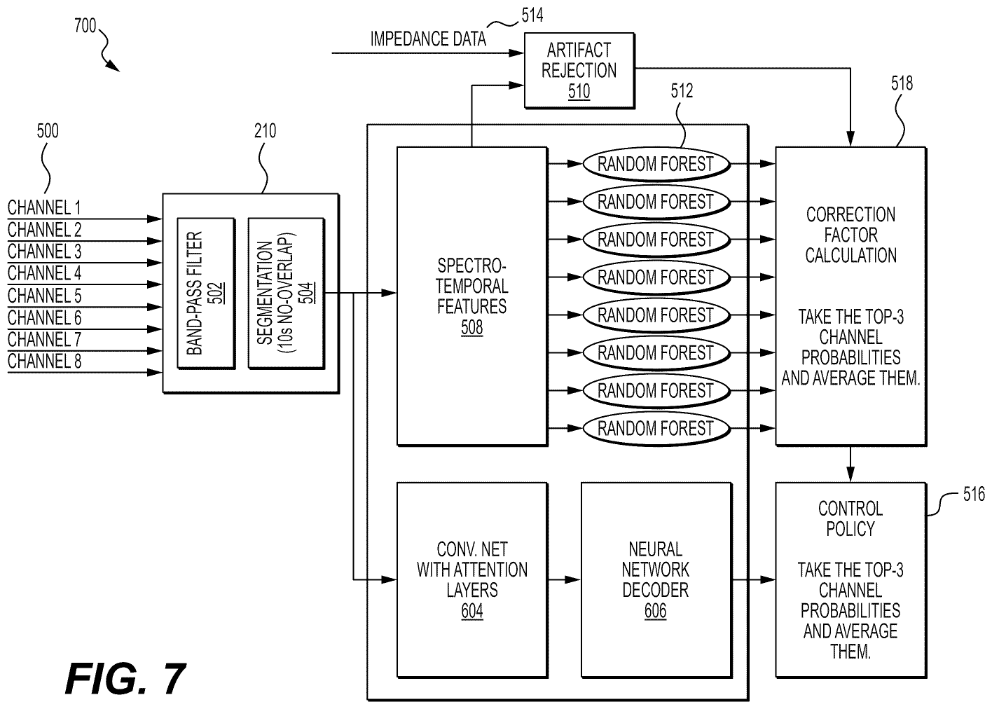

For example, the pathways/models 501 and 600 may be combined into a single, comprehensive model 700, as illustrated in FIG. 7. Referring to FIGS. 5-7, if the feature-based model 501 generates a first, low probability (e.g., a first output 520) for seizure on an EEG segment, the calculated correction factor 518 may be subtracted from the second probability (e.g., the second output 620) generated by the transform model 600. After correction, if the transform output is still above a predetermined threshold, the EEG segment may be classified as seizure-positive. The predetermined threshold may range from about 0.40 to about 0.70, including all values and sub-ranges therein. For example, the predetermined threshold may be a value of about 0.40, about 0.45, about 0.50, about 0.55, about 0.60, about 0.65, or about 0.70. In some variations, the control policy may also include calculating a seizure burden, as further described below. In one variation, a 5-minute moving average time window (including, e.g., 30 ten second EEG segments) may be used to calculate the seizure burden. When the seizure burden crosses 90% (e.g., greater than 4.5 minutes of seizure activity), an alarm may be sounded on the bedside for detected seizures. A combination of 10 minute moving average time window (e.g., 90% threshold) and 60 minute moving average time window (e.g., 20% threshold) may be used for generating the status epilepticus alerts.

In some variations, two differing feature-based classification models (e.g., memoryless and contextual classifiers) may provide first and second outputs, respectively, and the transform model may provide a third output. The first, second, and third outputs, along with any additional correction factors (e.g., from the spike detection module and/or artifact rejection model) may be aggregated via a decision model. The combined seizure classification output may then be compared to a control policy to determine a seizure burden.

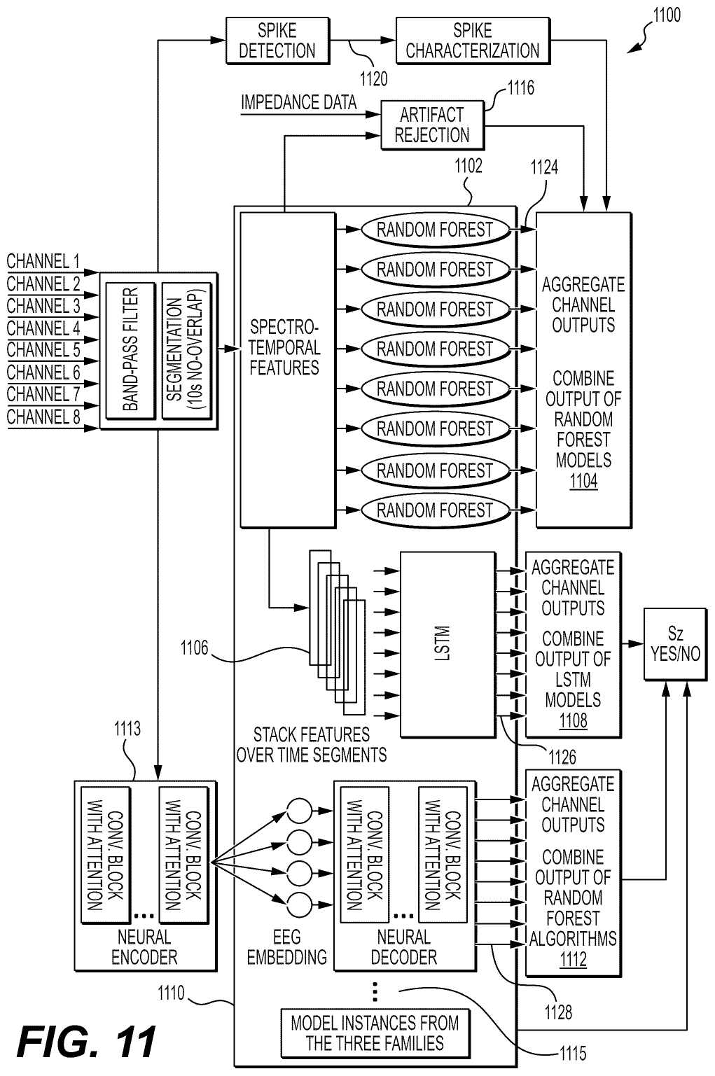

FIG. 11 illustrates a schematic of an exemplary seizure detection system 1100 that incorporates a plurality of parallel processing pathways for enhanced seizure detection accuracy. The system 1100 includes a first processing pathway comprising a first feature-based model 1102 implementing a memoryless classifier with a dedicated first decision model 1104 for aggregating channel outputs 1103. A second processing pathway employs a second feature-based model 1106 implementing a contextual classifier, with its own second decision model 1108 for aggregating channel outputs 1105. A third processing pathway utilizes a transform model 1110 with an encoder 1113 and decoder 1115 architecture, coupled with a third decision model 1112 for aggregating outputs 1111. The system further incorporates an artifact rejection model 1116 and a spike detection module 1120, which communicate with the feature-based models 1102/1106 and provide outputs 1118/1122 to the respective decision models 1104/1108. In some variations, the transform model 1110 may also receive inputs from the artifact rejection model 1116 and spike detection module 1120. The aggregated outputs 1124/1126/1128 from each pathway's decision model are combined to yield a final seizure classification 1130 for each temporal segment, which forms the basis for seizure burden calculation. This multi-pathway architecture enables the system to leverage complementary analysis approaches, with the memoryless classifier identifying distinctive features within individual segments, the contextual classifier capturing temporal patterns across segments, and the transform model processing raw EEG data directly to identify complex patterns that may not be captured by predefined feature sets.

### Specialized Models

The seizure detection system herein may, in some variations, comprise one or more specialized models configured to run in conjunction with (e.g., in parallel) or as a part of the seizure detection module. The one or more specialized models may be configured to address different classification or correction challenges in signal analysis. In some variations, one or more specialized models may be employed instead of one or more models described above (e.g., the classification model and/or the transform model). Exemplary variations of specialized models are further described below.

### (a) Symmetry Breaking Model

A first specialized model may be a symmetry breaking model trained to preferentially identify atypical or non-prototypical activity in EEG signals. The symmetry breaking model may be configured as a convolutional neural network (CNN) specifically trained to preferentially identify difficult-to-classify EEG patterns that may be missed by standard classification approaches. This atypical activity may be challenging to identify using standard classification approaches, and may include low-amplitude seizure patterns, evolving seizure onset patterns, brief epileptiform discharges, boundary-state electrographic activity, and/or seizure patterns with artifact-like characteristics.

The symmetry breaking model may receive the same pre-processed EEG data as the primary deep learning model. The model may be trained to focus specifically on EEG patterns that exist at classification boundaries where standard models may perform poorly. This specialization may come with a natural trade-off of potentially reduced performance on typical EEG classification tasks, but may significantly enhance the system's ability to detect challenging seizure patterns that might otherwise be missed.

In some variations, the symmetry breaking model may employ specialized loss functions during training that may assign higher weights to difficult-to-classify examples. These loss functions may include focal loss, weighted cross-entropy, or custom loss functions designed to emphasize boundary cases. The model architecture may incorporate attention mechanisms that help the model focus on subtle discriminative features that may be indicative of non-prototypical seizure activity. The depth of the convolutional neural network may range from, for example, about 2 to 20 layers.

The symmetry breaking model may generate probability outputs that may indicate the likelihood of seizure activity for each temporal segment. In some variations, this model may be configured to output probability values for each channel, with the highest probability channels being selected for further processing. The model may consider the top 2 channels (although alternatively, the top 1, top 3, or top N where N may range from 1 to the total number of channels may be employed) and calculate an average probability for seizure detection.

### (b) Sequence Breaking Model

A second specialized model may be a sequence breaking model configured to preferentially reduce consecutive classification errors. That is, the sequence breaking model may be configured to perform sequential error mitigation by analyzing temporal relationships between consecutive EEG segments. The sequence breaking model may be configured as a machine learning model (e.g., convolutional neural network (CNN)) trained to reduce consecutive classification errors across temporally adjacent segments. This model may be particularly valuable for ensuring temporal consistency in seizure classification, addressing scenarios where individual segments may be misclassified in a way that creates an implausible sequence of classifications.

The sequence breaking model may identify and correct classification inconsistencies based on learned patterns of electrographic evolution. For example, it may be unusual for a single short temporal segment to show seizure activity when both the preceding and following segments show no activity, or for a single non-seizure segment to appear between clear seizure segments. The sequence breaking model may be trained to recognize such implausible patterns and adjust classifications accordingly to maintain temporal coherence.

In some variations, the sequence breaking model may analyze sequences of temporal segments spanning from about 2 to 100 consecutive segments (e.g., about 3 to 50 segments, about 5 to 25 segments, or about 10 to 15 segments). This context window may allow the model to evaluate the plausibility of classification patterns over time periods ranging from about 20 seconds to several minutes, such as over 10 minutes.

The sequence breaking model may generate probability outputs similarly to the symmetry breaking model, considering the top 2 channels (or alternatively, the top 1, top 3, or top N channels) and calculating an average probability.

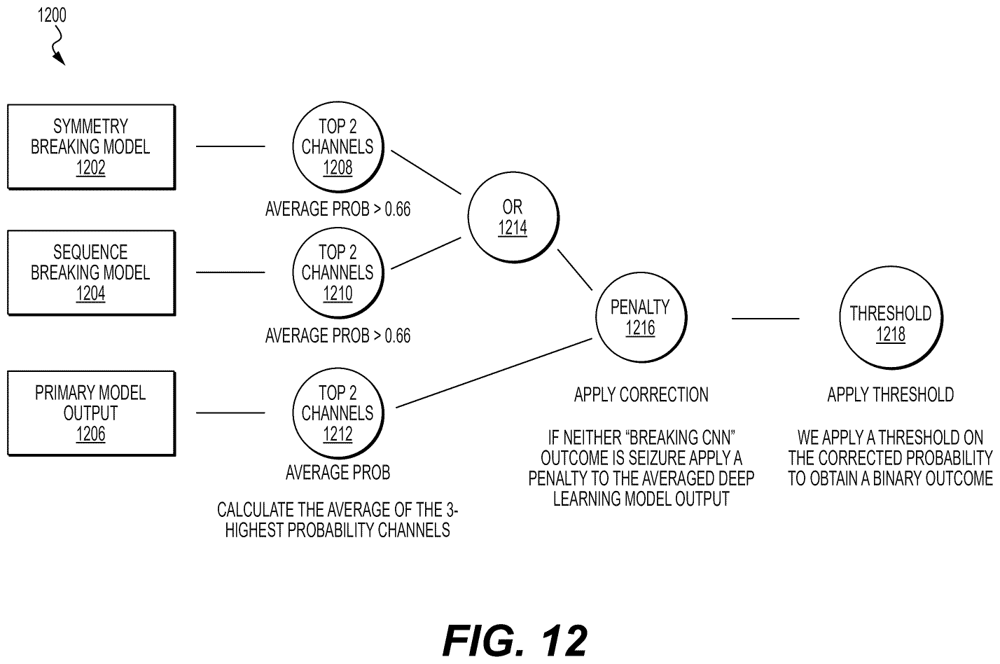

In some variations, the outputs from the specialized models may be integrated with the primary deep learning model output through a control policy, as will discussed herein with reference to FIG. 12.

### Seizure Burden Calculation and Output

### (a) Control Policy and Seizure Burden

The outputs from the feature-based and deep learning models, along with specialized models such as symmetry breaking and sequence breaking models, may be combined using a correction scheme to provide a seizure detection system having improved accuracy and processing speed. Seizure burden and/or insights bar detections (highlighting of potential areas of concern within an EEG trace) as further described below may be generated from combinations of the various outputs.

In some variations, the seizure classification may include classifying each temporal data segment for each channel as (1) seizure-positive or (2) seizure-negative. The seizure binary classification may use machine learning algorithms as described elsewhere herein. The method may include aggregating the seizure binary classifications for the plurality of temporal data segments for the plurality of channels over a moving time window. The aggregated seizure classifications may be subjected to a control policy module 275 of the seizure burden calculation and output module 270, as shown in FIG. 2. FIG. 2 shows the seizure burden calculation and output module 270. As shown in FIG. 2, the seizure burden calculation and output module 270 may comprise a control policy module 275, a seizure burden calculation module 280, a seizure burden plot module 285, and a seizure burden notification module 290. The control policy module 275 may be configured to implement a control policy, the seizure burden calculation module 280 may be configured to calculate a seizure burden value, the seizure burden plot module 285 may be configured to plot seizure burden values, and the seizure burden notification module 290 may be configured to provide notifications as described elsewhere herein, respectively.

In some cases, the moving analysis window may have a period of time between 1 minute and 1 hour. In some cases, the period of time of the moving analysis window may be dynamic or adjustable instead of fixed. In some cases, the period of time of the moving analysis window may be dependent on the subject.

In some variations, a cluster of seizure-positive classifications on one or more channels may be subjected to a control policy module 275 to result in an overall determination of a seizure for the patient for a corresponding time epoch.

The control policy may be a set of rules that result in an overall determination of seizure for the patient for a corresponding time epoch. The control policy may take a set of parameters as input and act on the set of parameters according to the set of rules to result in an overall determination of a seizure for the patient for a corresponding time epoch. The set of rules may be as described elsewhere herein. The set of rules may be adjusted at any point of time to act on more parameters or to act on less parameters. The set of rules may be adjusted at any point of time to include more rules or to remove rules. The set of rules may be at least about 1, 2, 3, 4, 5, 6 7, 8, 9, 10, 15, 20, 25, 50, 100, 500, 1000, or more rules. The set of rules may be at most about 1000, 500, 100, 50, 25, 20, 15, 10, 9, 8, 7, 6, 5, 4, 3, 2 or less rules. The set of rules may be from about 1 to 1000, 1 to 500, 1 to 100, 1 to 25, 1 to 10, 1 to 5, or 1 to 3 rules.

In some variations, the input of parameters for the control policy may include, the quantity of classification of channels as seizure-positive, the quantity of classification of channels as seizure-negative, the classification of channels as seizure-positive, the classification of channels as seizure-negative, the corresponding time epoch, the quantity of channels, the machine learning algorithm used for classification, a moving analysis window time length, the quality of the connection of each channel, information derived from EKG signals, information derived from EMG signals, information regarding the patient's demographics, information regarding the patient's current or previous condition, information regarding treatments or medications applied to the patient, information derived from movement sensors (e.g., an accelerometer or inertial measurement unit), etc.

In some variations, the control policy may combine outputs from multiple classification models to determine seizure classifications. As illustrated in FIG. 12, the control policy may receive inputs from a primary deep learning model, a symmetry breaking model trained to preferentially identify non-prototypical epileptiform activity, and a sequence breaking model configured to perform sequential error mitigation. The control policy may evaluate the outputs from these models by taking the top 2 or top 3 channel probabilities from each model and calculating an average probability for each model. The control policy may then apply a decision logic that may implement an OR operation to determine if at least one specialized model indicates seizure activity. If neither specialized model indicates seizure activity while the primary model does, a penalty or correction factor may be applied to the primary model's output. After applying any corrections, the control policy may compare the corrected probability to a threshold to obtain a binary seizure classification outcome.

The control policy may improve output accuracy while reducing computational requirements. In some variations, utilizing the control policy may comprise applying an adaptive channel selection policy. The adaptive channel selection policy may comprise selecting channels having highest probability values. The adaptive channel selection policy may be configured to determine signal quality measures for each channel, evaluate probability values associated with each channel, and select channels for further processing based on a combination of the signal quality measures and probability values.

In some variations, the adaptive channel selection policy may be configured to prioritize channels exhibiting strongest seizure-related characteristics. The channel selection criteria may be progressively refined across multiple implementations of the seizure detection method, wherein the refinement increases seizure detection confidence while reducing a required number of channels.

The control policy may function with any suitable number of channels, such as greater than or less than 4 channels (e.g., 2 or 3 topological channels). In some variations, the control policy may evaluate top-ranked channel probabilities and combine these probabilities to drive an overall detection. This approach may facilitate detection of seizure on fewer channels to drive an overall detection, which may be helpful for classifying short seizures.

In some variations, the control policy may have any number input of parameters. The control policy may have an input of at least about 1, 2, 3, 4, 5, 6, 7, 8, 9, 10, 11, 12, 13, 14, 15, 20, 25, 50, 100, 500 1000 or more parameters. The control policy may have an input of at most about 1000, 500, 100, 50, 25, 20, 15, 14, 13, 12, 11, 10, 9, 8, 7, 6, 5, 4, 3, 2 or less parameters. The control policy may have an input from about 1 to 1000, 1 to 500, 1 to 100, 1 to 50, 1 to 25, 1 to 15, 1 to 10, or 1 to 5 parameters.

In some variations, the set of rules may dictate that the control policy discards the classification of a channel. For example, if the control policy receives an input of a single a seizure-positive classification for a corresponding time epoch, the set of rules may discard the seizure-positive classification for the corresponding time epoch. In some cases, the control policy may receive at least about 1, 2, 3, 4, 5, 6, 7, 8, 9, 10, 50, 100, 500, or more seizure-positive classifications and the set of rules may discard each seizure-positive classification for the corresponding time epoch. In some cases, the control policy may receive at most about 500, 100, 50, 10, 9, 8, 7, 6, 5, 4, 3, 2 or less seizure-positive classifications and the set of rules may discard each seizure-positive classification for the corresponding time epoch. In some cases, the control policy may receive from about 1 to 500, 1 to 100, 1 to 50, 1 to 10, or 1 to 5 seizure-positive classifications and the set of rules may discard each seizure-positive classification for the corresponding time epoch.

In some variations, the set of rules may dictate that the control policy output a seizure-positive classification for a set of channels corresponding to a time epoch. For example, if the control policy receives a set of four or more channels that each register a seizure-positive classification for the corresponding time epoch, the set of rules may output a seizure-positive classification for the corresponding time epoch. In some cases, the control policy may receive a set of seizure-positive classifications of at least about 1, 2, 3, 4, 5, 6, 7, 8, 9, 10, 15, 20, 25, 50, 100, 1000 or more channels, the set of rules may output a seizure-positive classification for the set of seizure-positive classifications for the corresponding time epoch. In some cases, the control policy may receive a set of seizure-positive classifications of at most about 1000, 100, 50, 25, 20, 15, 10, 9, 8, 7, 6, 5, 4, 3, 2, or less channels, the set of rules may output a seizure-positive classification for the set of seizure-positive classifications for the corresponding time epoch. In some cases, the control policy may receive a set of seizure-positive classifications from about 1 to 1000, 1 to 500, 1 to 100, 1 to 50, 1 to 25, 1 to 10, or 1 to 5, the set of rules may output a seizure-positive classification for the set of seizure-positive classifications for the corresponding time epoch.

In some variations, the control policy may apply correction factors to adjust probability values before determining the final classification. For example, if the feature-based classifier generates a low first probability value, a correction factor may be subtracted from the second probability value generated by the transform model. Similarly, if neither specialized model (symmetry breaking or sequence breaking) indicates seizure activity, a penalty may be applied to the averaged output from the primary deep learning model. These correction mechanisms may help to reduce false positives by leveraging the complementary strengths of the different classification approaches.

As noted above, the outputs from the specialized models may be integrated with the primary deep learning model output through a control policy. The control policy may evaluate the outputs from all utilized models to determine the final seizure classification. When the symmetry breaking model or sequence breaking model indicates seizure activity, this information may be used to support a seizure-positive classification from the primary model. Conversely, if neither specialized model indicates seizure activity while the primary model does, a penalty or correction factor may be applied to the primary model's output.

In particular, the control policy may consider the average probability from the top channels of each model and apply a logical OR operation to determine if at least one model provides strong evidence of seizure activity. Alternatively, if neither specialized “breaking” CNN indicates seizure activity, a penalty may be applied to the averaged output from the primary deep learning model. After applying any corrections, a threshold may be applied to the corrected probability to obtain a binary outcome (seizure-positive or seizure-negative).

This integration approach may leverage the complementary strengths of the different models—the primary model's performance on typical patterns, the symmetry breaking model's ability to identify difficult cases, and the sequence breaking model's capacity to enforce temporal consistency. The combination of these models through the control policy may result in improved sensitivity and specificity for seizure detection, reducing both false positives and false negatives compared to any single model approach.

FIG. 12 is a flow chart of an exemplary method 1200 of classifying seizures using aggregated outputs from three deep learning models. The method 1200 may incorporate three parallel model pathways: a symmetry breaking model 1202, a sequence breaking model 1204, and a primary deep learning model 1206. Each model may process EEG data and generate probability outputs for seizure detection. The symmetry breaking model 1202 may be configured to identify non-prototypical epileptiform activity and may output an average probability 1208 based on the top 2 channels with highest probabilities. Similarly, the sequence breaking model 1204 may be configured to ensure temporal consistency across segments and may output an average probability 1210 based on its top 2 channels. The primary deep learning model 1206 may generate an average probability 1212 based on the top 3 highest probability channels. The outputs from these three models may be evaluated by a decision logic 1214 that may implement an OR operation to determine if at least one specialized model indicates seizure activity. If neither “breaking” CNN outputs indicate seizure, a penalty 1216 may be applied to the primary model output. The corrected probability may then be compared to a threshold 1218 to obtain a binary seizure classification outcome 1220. This integration of specialized models with the primary model may enable more robust seizure detection by addressing both difficult-to-classify patterns and ensuring temporal consistency in the classifications.

In some variations, the method may include calculating the seizure burden of the patient as the percentage of seizure-positive classifications within a specified period of time. As shown in FIG. 2, the seizure burden calculation module 280 may be configured to calculate the seizure burden of the patient. In some cases, the period of time used for seizure burden calculation may be between 1 minute and 1 hour. In some cases, the period of time used for seizure burden calculation may be the entirety of the recording session. In some cases, the period of time used for seizure burden calculation may be dynamic or adjustable instead of fixed.

In some variations, the seizure burden may form a continuous output measure by calculating seizure burden for a moving analysis window of time to result in a seizure burden value for individual sequential periods of time. In some cases, the period of time of the moving analysis window may be between 1 minute and 1 hour. In some cases, the period of time of the moving analysis window may be dynamic or adjustable instead of fixed. In some cases, the sequential periods of time formed by the moving analysis window may be overlapping. In some cases, sequential periods of time formed by the moving analysis window may be non-overlapping. In some cases, the moving analysis window may move in time increments between 1 second and 1 hour. In some cases, the moving analysis window may pause or skip periods of time such that the resulting seizure burden values are not continuous or not sequential in time.

In some variations, the seizure burden calculation may utilize multiple moving analysis windows of different durations to generate different types of alerts. For example, a 5-minute moving analysis window (e.g., 30 ten-second EEG segments) may be used to calculate the seizure burden for generating seizure alerts. When this 5-minute seizure burden crosses 90% (e.g., greater than 4.5 minutes of seizure activity), an alarm may be generated for detected seizures. A combination of a 10-minute moving analysis window with a 90% threshold and a 60-minute moving analysis window with a 20% threshold may be used for generating status epilepticus alerts. This multi-window approach may allow the system to detect both acute seizure events and longer-term seizure states that may require different clinical interventions.

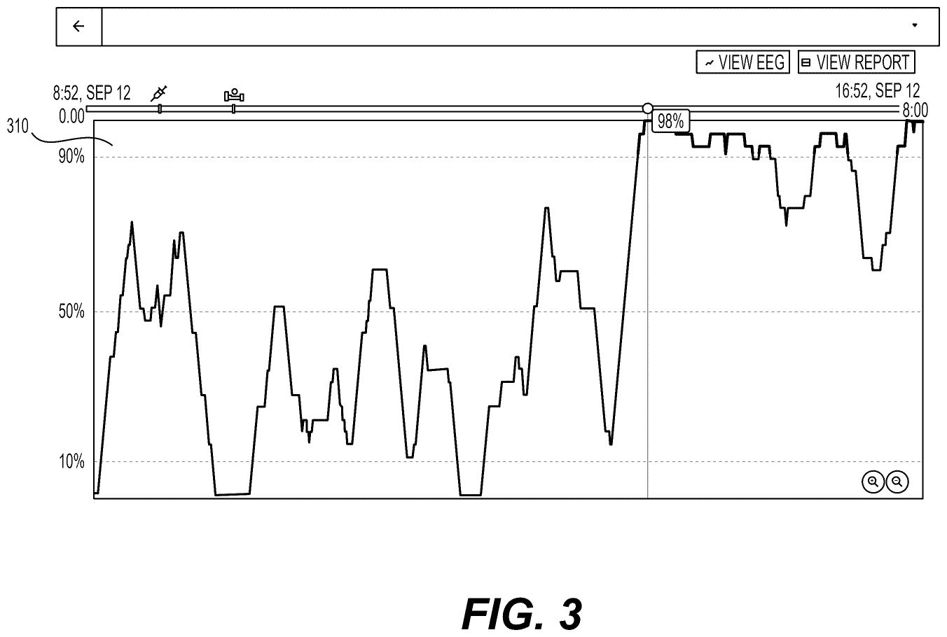

FIG. 2 shows a seizure burden plot module 285 configured to plot the seizure burden of a subject. As shown in FIG. 3, the seizure burden output may be displayed to the user as a time-series plot 310 where each point represents the seizure burden for a period of time. In some variations, the seizure burden output may display the one or more thresholds (e.g., 10%, 50%, 90%, etc.) to the user on the time-series plot. In some variations, the seizure burden output may be displayed to the user as a time-series plot, bar graph, or chart etc. In some variations, the time-series plot may be depicted in a certain color to note the threshold that has been passed. As shown in FIG. 3, when the time-series plot of the seizure burden passes the 50% threshold for a period of time, the time-series plot may change from gray to orange. In some cases, when the time-series plot passes 90%, the time-series plot may change from orange to red. The time-series plot may be of any color and the passing of a threshold may be illustrated in any color. In some cases, the seizure burden plot module may display a wide variety of information, for example, the time period measured, the date, or the initial time acquisition, etc. In some cases, the seizure burden plot may be usable by a healthcare practitioner to assess the condition of the subject and determine a course of treatment. The seizure burden plot may also be usable by a healthcare practitioner to monitor the progression of the subject's condition over time or to monitor the effectiveness of courses of treatment.

FIG. 2 shows a seizure burden notifications module 290 configured to generate notifications. In some variations, the method may include generating one or more notifications when seizure-positive classifications have been made or when the seizure burden value is equal to or exceeds one or more thresholds. When the seizure burden value is equal to or exceeds a threshold (e.g. the 90% threshold), the system may display to a subject (e.g., patient) or user (e.g., healthcare practitioner, doctor, nurse, etc.) a notification that the system has detected continuous seizure activity. The notification may also include any color. For example, the background of the screen displaying the notification may be red. The text of the notification may be any color, for example, white. The color of the background of the screen may correlate with the value of the seizure burden calculation. For example, if the seizure burden is equal to or above a certain threshold, the selected color for the background of the screen may indicate that the seizure burden is equal to or above a threshold. The color of the text of the notification may correlate with the value of the seizure burden calculation. For example, if the seizure burden is equal to or above a certain threshold, the selected color for the text of the notification may indicate that the seizure burden is equal to or above a threshold.

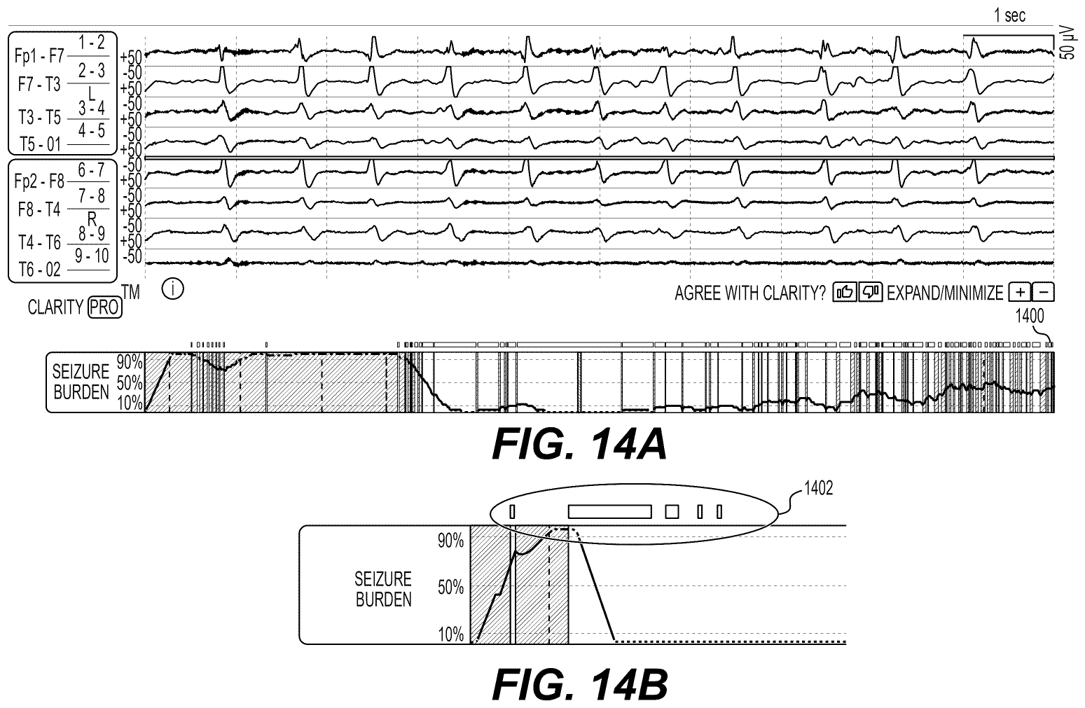

The system may also display a wide variety of information to the subject or user in addition to the notification of detected continuous seizure activity. The system may display the seizure burden plot, the percentage of seizure burden calculated, and/or the time period for which the continuous seizure activity was detected (e.g., 7:40 μm to 7:50 pm), etc. In some variations, as shown in FIG. 14A, the systems may be configured such that the output of the hybrid models described herein may be displayed as an insights bar 1400 that identifies areas of potential concern on an EEG trace, e.g., seizure activity or status epilepticus. An enlarged view of a portion of an insights bar 1402 is shown in FIG. 14B highlighting an area of the EEG trace where seizure burden has surpassed about 90%. The insights bar (1400, 1402) may help clinicians identify patients who may be experiencing seizures or other neurological issues quicker, allowing for faster intervention and thus, better outcomes.

The one or more notifications may be usable by a healthcare practitioner to assess the condition of the subject and determine a course of treatment. In some cases, one or more notifications may be generated when the seizure burden value is equal to or exceeds one or more thresholds as described elsewhere herein. In some cases, the one or more notifications may be generated in the form of visual, audio, and/or textual alerts. The device may include speakers to provide audio notifications. In some cases, the one or more notifications may be delivered via networked communication technology such as the internet, telephone, facsimile, pager, short message service, etc. In some cases, the form, content, or delivery mechanism of the one or more notifications generated may depend on the seizure burden value. In some cases, the user may be able to select the form, content, or delivery mechanism of the one or more notifications generated.

In some variations, the seizure burden calculation module may calculate a seizure burden value. The seizure burden notification module may output a notification if the seizure burden value crosses a threshold value. For example, if the seizure burden value crosses a threshold of 10%, a notification may be generated. In another example, if the seizure burden value crosses a threshold of 50%, a notification may be generated. In another example, if the seizure burden value crosses a threshold of 90%, a notification may be generated. In some cases, a first notification indicative of frequent seizure activity may be generated when the seizure burden is equal to or exceeds a first threshold of 10%. In some cases, a second notification indicative of abundant seizure activity may be generated when the seizure burden is equal to or exceeds a second threshold of 50%. In some cases, a third notification indicative of continuous seizure activity may be generated when the seizure burden is equal to or exceeds a third threshold of 90%. In some cases, notifications may be generated to a specific person that the method is programmed to notify.

In some variations, the threshold for notification may be of any percentage. The threshold for notification may be at least about 1%, 5%, 10%, 15%, 20%, 25%, 30%, 35%, 40%, 45%, 50%, 55%, 60%, 65%, 70%, 80%, 90%, 95%, 99% or more. The threshold for notification may be at most about 99%, 95%, 90%, 85%, 80%, 75%, 70%, 65%, 60%, 55%, 50%, 45%, 40%, 35%, 30%, 25%, 20%, 15%, 10%, 5%, 1% or less. The threshold for notification may be from about 0% to 100%, 1% to 99%, 5% to 95%, 10% to 90%, 20% to 80%, 30% to 70%, or 40% to 60%. The threshold for notification may also be user adjustable.

In some variations, the seizure burden notification module may have any number of thresholds. The seizure burden notification module may have at least about 1, 2, 3, 4, 5, 6, 7, 8, 9, 10, 15, 20, 25, 50, 100, 500, 1000, 5000 or more thresholds. The seizure burden notification module may have at most about 5000, 1000, 500, 100, 50, 25, 20, 15, 10, 9, 8, 7, 6, 5, 4, 3, 2 or less. The seizure burden notification module may have from about 1 to 5000, 1 to 1000, 1 to 500, 1 to 100, 1 to 50, 1 to 25, 1 to 10, or 1 to 5 thresholds. The quantity of thresholds may be increased or decreased at any point in time.

In some variations, the seizure burden notification module may provide any number of notifications. The seizure burden notification module may provide at least about 1, 2, 3, 4, 5, 6, 7, 8, 9, 10, 15, 20, 25, 30, 35, 40, 50, 100, 500, 1000 or more notifications. The seizure burden notification module may provide at most about 1000, 500, 100, 50, 40, 35, 30, 25, 20, 15, 10, 9, 8, 7, 6, 5, 4, 3, 2 or less notifications. The seizure burden notification module may provide from about 1 to 1000, 1 to 500, 1 to 100, 1 to 50, 1 to 25, 1 to 10, or 1 to 5 notifications.

In some variations, the seizure burden calculation and output module may criteria in addition to the seizure burden threshold to output a notification. In some cases, the duration of time that seizure burden is over a threshold may be used to determine if a notification is output. In some cases, amount of time since the previous crossing of a seizure burden threshold may be used to determine if a notification is output. In some cases, dynamic criteria may be applied with a combination of time based, seizure burden based, and other policies to determine if a notification is output.

In some variations, the system may be coupled with other systems. In some cases, the systems may be eye trackers, movement sensors (e.g., an accelerometer or an inertial measurement unit), electromyography (EMG), electrocardiogram (ECG or EKG), etc.

### Model Training Approaches

Different classes of models within the seizure detection system may be trained using distinct approaches optimized for their specific functions and architectures. These training methodologies may enable each model to develop specialized capabilities while ensuring robust performance across diverse patient populations and EEG patterns.

For feature-based models, a memoryless training approach may be employed in which each temporal segment is evaluated independently during training without consideration of adjacent segments. This training process may involve at least about 5,000 hours (e.g., about 5,000 to about 80,000 hours or about 10,000 to about 50,000 hours) of annotated EEG data from at least about 500 subjects (e.g., about 500 to about 8,000 subjects or about 1,000 to about 4,000 subjects). The training data may comprise at least about 1,000,000 labeled temporal segments (e.g., about 1,000,000 to about 50,000,000 segments or about 2,000,000 to about 20,000,000 segments). The memoryless training approach may allow the model to focus on discriminative features present within individual segments rather than relying on temporal patterns. Feature importance may be calculated during training to identify the most predictive features for seizure detection, and these importance scores may be used to prioritize certain features during inference.

For deep learning models, such as the transform model with encoder-decoder architecture, a more comprehensive training approach may be required. The transform model may be trained using at least about 10,000 hours (e.g., about 10,000 to about 150,000 hours or about 25,000 to about 100,000 hours) of EEG data from at least about 1,000 subjects (e.g., about 1,000 to about 12,000 subjects or about 2,000 to about 8,000 subjects). This may correspond to at least about 3,000,000 temporal segments (e.g., about 3,000,000 to about 80,000,000 segments or about 5,000,000 to about 40,000,000 segments). The training process may involve multiple stages, including pre-training on a large corpus of unlabeled EEG data to learn general patterns, followed by supervised fine-tuning on labeled seizure and non-seizure data. Transfer learning techniques may be employed to adapt pre-trained models to new subjects with limited subject-specific data, wherein most of the model parameters are preserved from the pre-training phase while a subset of parameters, typically in the later layers of the network, are fine-tuned for the new subject.

Contextual classifiers, including LSTM-based models, may be trained using a context-dependent approach where temporal relationships between adjacent segments are explicitly considered. Training may involve exposing the model to sequences of at least about 3 to about 30 consecutive temporal segments, allowing it to learn patterns that evolve over time. The training data may comprise at least about 8,000 hours (e.g., about 8,000 to about 120,000 hours or about 15,000 to about 70,000 hours) of EEG data from at least about 800 subjects (e.g., about 800 to about 10,000 subjects or about 1,500 to about 6,000 subjects), with particular emphasis on capturing diverse seizure evolution patterns and transitions between seizure and non-seizure states.

For specialized models such as the symmetry breaking and sequence breaking models, a context-dependent training approach may be employed with specific modifications to address their unique objectives. As previously noted, at least about 1,000 hours (e.g., about 1,000 to about 100,000 hours or about 20,000 to about 60,000 hours, such as about 40,000 hours) of EEG data may be aggregated from at least 500 subjects (e.g., 500 to 10,000 patients or 1,000 to 5,000 patients, such as 3,000 patients) and compiled for model training. For the symmetry breaking model, the training process may involve oversampling difficult-to-classify boundary cases and employing specialized loss functions that assign higher weights to these challenging examples. This focused training may enable the model to develop sensitivity to non-prototypical epileptiform patterns that might be missed by standard classifiers. For the sequence breaking model, training may emphasize temporal consistency by presenting the model with artificially created sequences containing implausible transitions between seizure and non-seizure states, training it to identify and correct such inconsistencies.

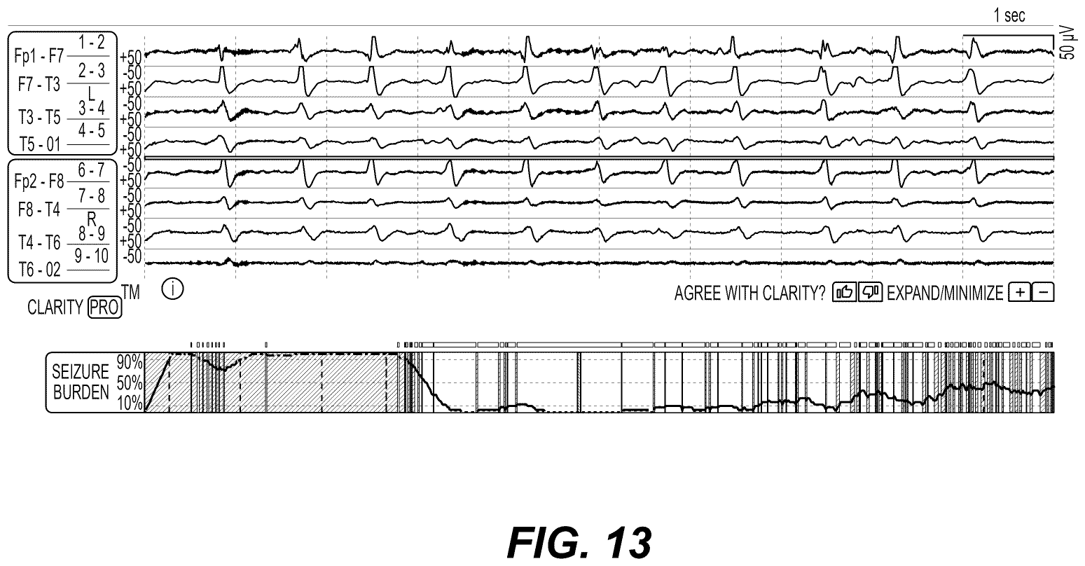

FIG. 13 provides an exemplary annotation software interface used for data collection and annotation. The upper portion displays multi-channel EEG recordings with a dropdown menu showing different event type options (e.g., seizure, drowsy, all left, 1-2 (Fp1-F7), 2-3 (F7-T3), 3-4 (T3-T5), etc.) that expert annotators may select to identify specific patterns within the raw EEG traces. The interface allows annotators to mark spatial-temporal annotations directly on the EEG recordings. The lower portion displays a seizure burden plot that visualizes how the calculated seizure burden changes over time, with color-coding indicating different severity levels. This annotation software facilitated the collection of the extensive dataset used for model training, which included 40,000 hours of EEG data aggregated from 3,000 patients. The collected data was used to train models in two distinct ways: (1) memoryless training for random forest models and end-to-end CNN models, where each model was exposed to the annotation from a current 10s window of EEG during evaluation; and (2) context-dependent training for specialized models like the symmetry breaking CNN and sequence breaking CNN, where weighted combinations from different patients and time-windows were pooled together to facilitate learning of long-term patterns.

### Computer Systems

The present disclosure provides computer systems that are programmed to implement methods of the disclosure, including the control of the seizure detection system, control hardware components, receive and process data, interface with a user, etc. A seizure detection system and its various components may include computer hardware (and associated firmware) that may be electrically connected to a computer control system. A control unit may include such a computer system.

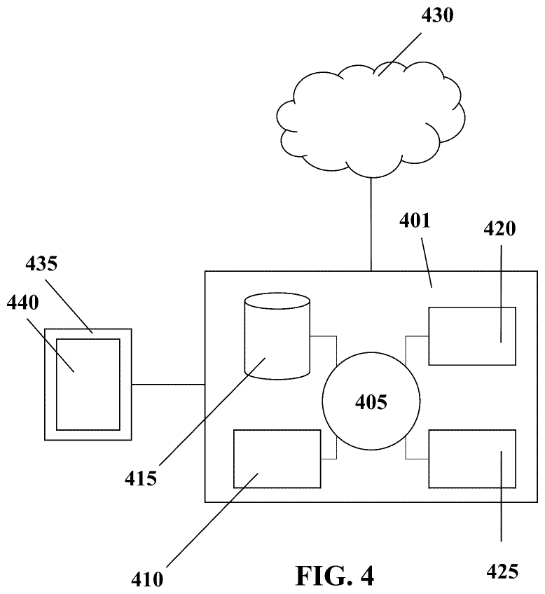

FIG. 4 shows a computer system 401 that is programmed or otherwise configured to operate and/or control the EEG device module and the seizure detection module. The computer system 401 can regulate various aspects of the seizure detection system of the present disclosure, such as, for example, processing EEG signals, segmenting EEG signals, extracting features, processing features with machine learning algorithms, implementing the control policy and seizure burden, calculating the seizure burden value, plotting the seizure burden, providing notifications, etc. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., seizure detection system manager, seizure detection system user, seizure detection data acquirer, seizure detection system scribe, etc.). Examples of remote computer systems include servers, personal computers (e.g., portable PC), slate or tablet PC's (e.g., Apple® iPad, Samsung® Galaxy Tab), telephones, Smart phones (e.g., Apple® iPhone, Android-enabled device), or personal digital assistants. The user can access the computer system 401 via the network 430. In some cases, the EEG device module and the seizure detection module will be located within the same computer system. In some cases, the EEG device module will be located within one computer system which is networked to a remote computer system containing the seizure detection module. After performing seizure detection, the remote computer system can then transmit seizure detection results to the computer system containing the EEG device module as well as other remote computer systems that may be used for seizure detection results display. In some cases, different parts of the seizure detection module may be located in different computer systems which have been networked together.

Methods as described herein can be implemented by way of machine (e.g., computer processor) executable code stored on an electronic storage location of the computer system 401, such as, for example, on the memory 610 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 605. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, a login screen for an administrator to access software programmed to control the seizure detection system and functionality and/or for providing the operation status health of the seizure detection system. Examples of UI's include, without limitation, a graphical user interface (GUI) and web-based user interface.

Methods and systems of the present disclosure can be implemented by way of one or more algorithms, e.g., the models/classifiers described above. An algorithm may be implemented by way of software upon execution by the central processing unit 405. The algorithm can, for example, be component of software described elsewhere herein and may modulate the seizure detection system parameters (e.g., processing EEG signals, machine learning algorithms, control policy, seizure burden, notifications, etc.).

### EXAMPLES

The following examples are illustrative only and should not be construed as limiting the disclosure in any way.

### Example 1

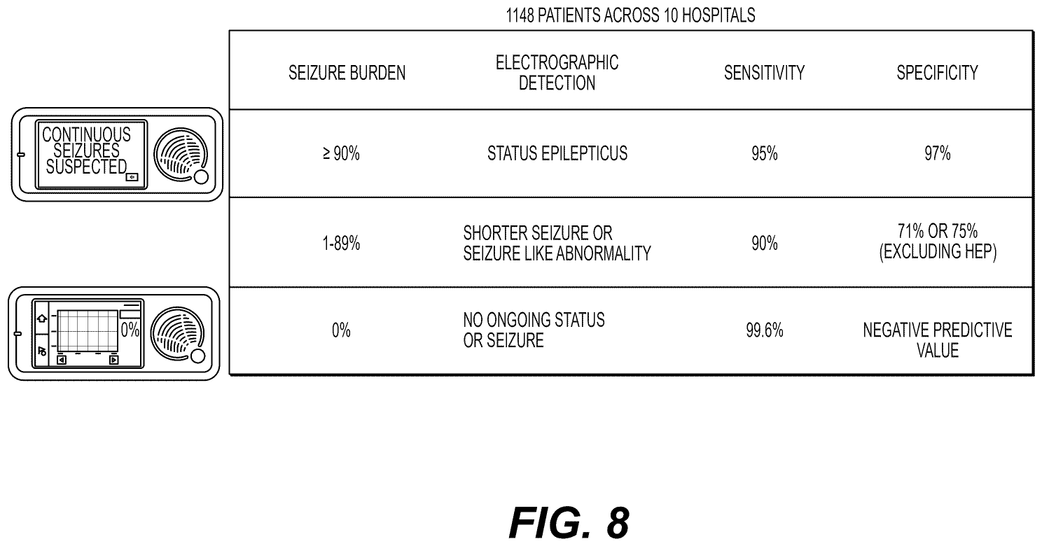

A hybrid model as described herein was used to analyze EEG data from 10 hospital sites, and included 1148 subjects. More specifically, the hybrid model included a feature-based model and a deep learning model. The feature-based model used a random forest machine learning model to extract features from EEG segments. The deep learning model used a convolutional neural network with attention layers to concurrently process the EEG data. As shown in FIG. 8, the output from the hybrid model was highly accurate. For example, the hybrid model was able to determine status epilepticus (e.g., a seizure burden of greater or equal to about 90%), with a sensitivity of about 95% and a specificity of about 97%. Shorter seizures or seizure-like abnormalities (e.g., a seizure burden of about 1-89%) were able to be determined with about 90% sensitivity, and between about 71% to about 75% specificity (excluding HEP). Additionally, the hybrid model was able to determine the absence of seizure activity (e.g., 0% seizure burden) with about 99.6% sensitivity.

When compared to a system running a single model, e.g., a machine learning model using a random forest classifier), the hybrid model resulted in improvements in sensitivity and specificity for seizure detection. For example, the sensitivity ranged between about 86% to about 95%, and there was an increase in status alerts of about 10%, a decrease in false alerts of about 22%, and a decrease in missed seizures of about 57%.

### Example 2

Another iteration of a hybrid model incorporating the spike detection module and specialized deep learning models (symmetry breaking CNN and sequence breaking CNN) alongside the models utilized in Example 1 was evaluated on an independent validation dataset consisting of 1875 subjects. This hybrid model demonstrated significant improvements over the model described in Example 1. For status epilepticus detection (seizure burden ≥90%), sensitivity increased by 38% while maintaining high specificity of 0% change. For shorter seizures or seizure-like abnormalities (seizure burden of about 1-89%), sensitivity improved by 22% and specificity improved by 2%.

When compared to a system without the spike detection module or specialized models, this hybrid model resulted in significant operational improvements, including an increase in status alerts of about 10%, a decrease in false alerts of about 22%, and a decrease in missed seizures of about 57%. These improvements demonstrate the value of increased artifact evaluation and incorporating specialized models trained to address specific classification challenges.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A system for analyzing EEG signals comprising:
a data module configured to receive data comprising a plurality of electroencephalography (EEG) signals recorded during a time window from a subject; and
a seizure detection module, the seizure detection module configured to process the data by dividing each of the plurality of EEG signals into a plurality of temporal segments, and comprising:
a feature-based classifier configured to:
extract a plurality of features from each of the plurality of temporal segments and generate a first probability value for each of the temporal segments derived from the plurality of extracted features; and
generate a correction factor based on the first probability value;
an encoder configured to transform the data into a plurality of vectors; and
a decoder configured to determine a second probability value based on the plurality of vectors,
wherein one or more processors is configured to determine one or more types of seizure activity based on the second probability value and the correction factor.

2. The system of claim 1, wherein the one or more processors is further configured to subtract the correction factor from the second probability value.

3. The system of claim 2, wherein the one or more processors is configured to subtract the correction factor from the second probability value when the first probability value is below a predetermined threshold.

4. (canceled)

5. The system of claim 1, wherein the one or more types of seizure activity comprises seizures, seizure-like activity, electrographic seizure, status epilepticus, post-ictal activity, highly pathological EEGs with a high likelihood of epileptiform activity, and an abnormal EEG pattern.

6. The system of claim 1, further comprising diagnosing epilepsy in the subject based on the detected seizure activity.

7. The system of claim 1, wherein at least one of the encoder and the decoder are part of a convolutional neural network.

8.-10. (canceled)

11. The system of claim 1, wherein the data module is configured to record the plurality of electroencephalography (EEG) signals over one or more channels.

12.-15. (canceled)

16. The system of claim 1, wherein a duration of the time window ranges from about 1 second to about 10 minutes.

17.-24. (canceled)

25. The system of claim 1, further comprising a headband, the headband comprising a plurality of electrodes from which the plurality of electroencephalography (EEG) signals is recorded.

26. The system of claim 1, wherein the seizure detection module further comprises a spike detection module configured to:
identify a spike within each of the plurality of temporal segments;
classify the spike based on one or more spike parameters; and
characterize each of the plurality of temporal segments as a physiological temporal segment or an artifactual temporal segment based on one or more spike metrics.

27. The system of claim 26, wherein the one or more spike parameters comprises a spike amplitude, a spike width, a spike prominence, or a spike polarity.

28. The system of claim 26, wherein the one or more spike metrics comprises a spike frequency, a spike jitter, a spike polarity, or a spike count.

29. The system of claim 26, wherein the seizure detection module is further configured to classify a temporal segment as an artifact when an impedance associated with an electrode used to record the EEG signals surpasses a threshold.

30. The system of claim 1, wherein the seizure detection module further comprises a contextual classifier configured to generate a plurality of context-based probability values based on a plurality of feature vectors extracted from a context window spanning a plurality of temporal segments.

31. The system of claim 30, wherein the contextual classifier comprises a recurrent neural network.

32. The system of claim 30, wherein the plurality of temporal segments within the context window comprises about 3 to about 30 temporal segments.

33. The system of claim 1, wherein the encoder comprises one or more attention blocks.

34. The system of claim 1, wherein the seizure detection module further comprises a decision module configured to combine two or more seizure probability values generated by a plurality of classifiers and correction factors to generate an aggregated probability value for each temporal segment.

35. The system of claim 34, wherein the aggregated probability value corresponds to a seizure burden and is determined by one or more of a mean, a median, and a maximum of the seizure probability values generated by the plurality of classifiers.

36. The system of claim 34, wherein the plurality of classifiers comprises at least the feature-based classifier, a contextual classifier, and the decoder.

37. The system of claim 34, wherein the decision module is further configured to:
compare a first moving average calculated over a first analysis window to a first threshold; and
generate a seizure alert when the first moving average exceeds the first threshold.

38. The system of claim 37, wherein the first moving analysis window is about 5 minutes and the alert is generated when the seizure burden exceeds about 90%.

39. The system of claim 37, wherein the decision module is further configured to:
compare a second moving average calculated over a second time window to a second threshold;
compare a third moving average calculated over a third analysis window to a third threshold; and
generate a status epilepticus alert when both the second moving average exceeds the second threshold and the third moving average exceeds the third threshold.

40. The system of claim 39, wherein the second analysis window is about 10 minutes, the third moving analysis window is about 60 minutes, the second threshold is about 90%, and the third threshold is about 20%.

41. A method for detecting seizures comprising:
receiving data from a plurality of electrodes, the data comprising a plurality of electroencephalography (EEG) signals recorded from a subject during a time window; and
using a seizure detection module configured to process the data, wherein the processing comprises:
dividing each of the plurality of EEG signals into a plurality of temporal segments;
extracting a plurality of features from each of the temporal segments;
determining a first probability value for each of the temporal segments derived from the plurality of extracted features, and generating a correction factor based on the first probability value;
transforming the processed data into a plurality of vectors, and determining a second probability value based on the plurality of vectors; and
determining a seizure classification for each of the temporal segments based on the second probability value and the correction factor.

42.-92. (canceled)
